(19) United States
(12) Patent Application Publication
(10) Pub. No.: US 2005/0203029 A1
(43) Pub. Date: Sep. 15, 2005

(54) Agents for treating <I>flaviviridae</I>infections

(76) Inventors: Ulrich Schubert, Uhlstadt (DE); Hans Will, Hamburg (DE); Huseyin Sirma, Elmshorn (DE)

Correspondence Address: STROOCK & STROOCK & LAVAN LLP, 180 MAIDEN LANE, NEW YORK, NY, 10038, US

(21) Appl. No.: 10/510,355

(22) PCT Filed: Apr. 4, 2003
(86) PCT No.: PCT/DE03/01213

§ 371 Date: Apr. 28, 2005

(30) Foreign Application Priority Data
Apr. 5, 2002 (DE) 102 16 227.1

(51) Int. Cl. A61K 31/366; A61K 38/04; A61K 31/69

(52) U.S. Cl. 514/19; 514/460; 514/64

(57) ABSTRACT

The invention relates to agents for the treatment, therapy and inhibition of Flaviviridae virus infections, which agents comprise proteasome inhibitors as the active component. The agents which are used for inhibiting the release, maturation and replication of Flaviviridae comprise, as the active component in pharmaceutical preparations, substance classes which share the common property of inhibiting the 26S proteasome in cells. These substance classes include, in particular, proteasome inhibitors which affect the activities of the ubiquitin/proteasome pathway, in particular the enzymic activities of the 26S and the 20S proteasome complex. The application of the invention lies in the antiviral therapy of Flaviviridae infections, especially in preventing the establishment and maintenance of a chronic hepatitis C virus infection and a hepatopathogenesis which is associated therewith.

The invention relates to agents (proteasome inhibitors) for the prevention, treatment, therapy and inhibition of the diseases caused in humans and animals by various members of the virus family Flaviviridae (Genera: Flavivirus, Pestivirus, Hepacivirus). This concerns, in particular, the diseases caused by hepaciviruses (hepatitis C viruses or HCVs; list of abbreviations given after the examples), especially hepatitis in humans. In addition to this, it concerns the diseases caused by pestiviruses, such as BVDV (bovine viral diarrhea virus) and CSFV (classical swine fever virus) in animals and the diseases caused by flaviviruses, such as West Nile virus, dengue virus and early summer meningoencephalitis (FSME) virus in humans and animals. Use of the agents according to the invention in pharmaceutical preparations leads principally or exclusively to the release of non-infectious viruses from infected cells. The same agents are able to restrict the spread of an acute infection both in cell cultures and in the infected body since all progeny viruses which are produced during treatment with these agents are non-infectious or virtually non-infectious. Furthermore, the agents are less toxic for non-proliferating hepatocytes and nonparenchymatous cells of the liver than they are for hepatoma cells. Consequently, these agents are suitable for preferentially destroying liver carcinoma cells, for example hepatocellular carcinomas (HCCs) in HCV-infected patients. The agents consist of a variety of substance classes which share the common property of inhibiting the 26S proteasome in cells. Taking as an example representatives of the genera Flavivirus and Pestivirus, it is shown that these agents, i.e. the proteasome inhibitors, drastically reduce the release of infectious viruses from infected host cells. In this connection, the pestivirus BVDV serves, due to its pronounced homology, both as regards the structure of the virus particle and as regards genomic organization, as a surrogate model for HCV, for which no infectious cell system is available. The antiviral therapy of hepatitis infections, especially for the purpose of preventing the establishment and maintenance of an acute and chronic HCV infection, represents the most striking example of applying this invention. Other application examples relate to treating BVDV infections in cattle and CSFV infections in pigs. In addition to this, these agents can also be used for preventing and treating infectious diseases which are caused by flaviviruses such as West Nile virus, yellow fever virus, dengue virus and early summer meningoencephalitis (FSME) virus. According to the present state of knowledge, it is to be assumed that proteasome inhibitors can suppress the viremia which is associated both with a fresh infection and with chronic infections and increase the success which is achieved in using the body's own immune system and/or known agents having a similar or different effect to eliminate the virus. Use of proteasome inhibitors can, for example, prevent, reduce or reverse the consequences of an HCV infection, such as liver damage of differing degrees of severity through to the development of hepatic cirrhosis/fibrosis or of an hepatic carcinoma.

Characterization of the Prior Art

0. Introduction

Infection with hepatitis C virus (HCV) is one of the greatest health problems world-wide (approx. 3% of the world population is affected). The most important feature of HCV infections is the chronic virus carrier status of the affected individuals, with this carrier status frequently lasting throughout life: more than 70% of HCV-infected individuals develop chronic hepatitis, which can in turn lead to chronically active hepatitis of varying degrees of severity through to hepatic cirrhosis and fibrosis (10-20% of all cases). Independently of this, primary hepatocellular carcinomas (HCCs; for reviews, see Lindenbach and Rice, 2001 and Major et al., 2001) develop in from 1 to 5% of chronically infected patients. In addition to this, HCV infections have been causatively connected to more than 30 other diseases in humans, including important autoimmune diseases such as type II cryoglobulinemia (mixed type II cryoglobulinemia, MC; Agnello, 2000). As a rule, HCV-infected patients are treated with a combination of interferon alpha and ribavirin. However, this method suffers from powerful side effects and only leads to elimination of the virus from the body in about half of all cases. This unsatisfactory situation is due, in the first place, to what is in general the broad diversity of HCV variants: at present, six genotypes are distinguished; these genotypes can in turn be diversified into a number of subtypes and strains (for a review, see Bukh et al., 1995). In the second place, a large number of new genetic variants develop in the chronically infected individuals. The genetic variability of HCV is probably also the cause of the inefficient immune response which can be observed in the majority of HCV-infected patients. For this reason, it has not previously been possible to develop either prophylactic or therapeutic vaccination methods. The passive administration of HCV-specific neutralizing antibodies and/or medicaments to patients who have received a liver transplant frequently does not prevent the transplanted liver being freshly infected, either. Immunosuppression in patients in whom hepatitides are subsiding or who have received a liver transplant can lead to viruses which are present latently being reactivated. It is necessary to develop novel therapeutic approaches in order to circumvent the problems of the antiviral and therapeutic agents for treating hepatitis C which have thus far been available. In the light of the variability of the virus, the methods which are of particular importance in this connection are those which exert an effect on conserved cellular factors which are essential for the multiplication of these viruses in the host cell. This invention describes such agents. Since neither cultured cells which can be infected efficiently nor a practicable animal model is presently available in the case of HCV, studies in this regard have been carried out using the pestivirus BVDV, which is related to HCV. The similarity of the BVDV and HCV life cycles has been demonstrated in a number of previous studies (for a review, see Lindenbach and Rice, 2001). In addition to this, experiments have been carried out using another, phylogenetically more distant, representative of the Flaviviridae family, i.e. the West Nile flavivirus. The invention relates to the surprising finding that inhibitors of the cellular 26S proteasome prevent infectious viruses being produced both from BVDV-infected and West Nile virus-infected cells. The homologous effect of proteasome inhibitors on the multiplication of pestiviruses and flaviviruses implies that these agents have a similar mechanism of action in all representatives of the Flaviviridae family; the reason for this is probably that Flaviviridae virions have homologous structures. It has been found that, in addition to the antiviral effect, the cytotoxic effect of proteasome inhibitors is significantly more strongly pronounced in hepatoma cells (liver cancer cells), which are dividing rapidly, than it is in slowly dividing primary hepatocytes. Because of these properties, proteasome inhibitors are particularly suitable for treating liver diseases and liver carcinomas which are due to HCV. The effect of proteasome inhibitors on pestiviruses suggests the possibility of using these agents in controlling the widely distributed animal pathogens BVDV (cause of bovine viral diarrhea and bovine mucosal disease, MD) and CSFV (cause of swine fever, (for a review see Thiel, 1996). The effect of proteasome inhibitors on the multiplication of flaviviruses opens up the possibility of using them in connection with a large number of diseases caused by flaviviruses in humans and in animals, including fevers, encephalitides and hemorrhages. The dengue fever virus, against which there have previously only been vaccines and forms of treatment which are inadequate, is of particular importance in this context (for a review, see Burke and Monath, 2001).

1. Function of the Ubiquitin/Proteasome Pathway

Proteasomes constitute the main proteolytic component in the nucleus and cytosol of all eukaryotic cells. They are multi-catalytic enzyme complexes which account for approximately 1% of all cell proteins. Proteasomes exercise a vital role in a wide variety of functions in cell metabolism. Their main function is that of the proteolysis of incorrectly folded, nonfunctional proteins and the rapid breakdown of regulatory proteins. The proteasomal breakdown of cellular or viral proteins has a further function for the T cell mediated immune response, by generating peptide ligands for major histocompatibility class I molecules (for a review, see Rock and Goldberg, 1999). Proteasome targets are as a rule labeled for breakdown by having oligomeric forms of ubiguitin (Ub) attached to them. Ub is a highly conserved protein which is 76 amino acids in length and is coupled covalently to target proteins. The ubiquitinylation is itself reversible, and a large number of Ub hydrolases can remove Ub molecules once again from the target molecule. The link between the ubiquitinylation of target proteins and proteasomal proteolysis is generally termed the ubiquitin/proteasome system (UPS) (for a review, see Rock and Goldberg, 1999; Hershko and Ciechanover, 1998).

The 26S proteasome is a multienzyme complex which is 2.5 MDa in size and which consists of approx. 31 subunits. The proteolytic activity of the proteasome complex is implemented by a cylindrical core structure, i.e. the 20S proteasome, which is 700 kDa in size and which consists of four superimposed rings. The 20S proteasome forms a complicated multienzyme complex which consists of 14 nonidentical proteins and which is arranged in two α rings and two β rings in an αββα sequence. The substrate specificity of the 20S proteasome encompasses three important activities: trypsin-, chymotrypsin- and postglutamyl peptide-hydrolyzing (PGPH) or caspase-like activities, which are located in the Z, Y and Z β-subunits. The 20S proteasome degrades in vitrodenatured proteins independently of their polyubiquitinylation. By contrast, in vivo enzymic activities of 20S proteasome are regulated by the addition of the 19S regulatory subunits, which together form the active 26S proteasome particle. The 19S regulatory subunits are involved in recognition of polyubiquitinylated proteins and in the unfolding of target proteins. The activity of the 26S proteasome is ATP-dependent and almost exclusively only degrades polyubiquitinylated proteins (for a review, see Hershko and Ciechanover, 1998).

1.1. Significance of the UPS in the pathogenesis of Clinically Relevant Diseases

The central role of the UPS in the cell explains the importance of this system for a large number of pathological phenomena (for a review, see Ciechanover et al., 2000). For example, the level of the tumor suppressor protein p53 is extremely low in particularly aggressive forms of cervical carcinomas which are induced by certain isolates of the human papilloma virus (HPV). HPV oncoprotein E6 induces breakdown of the suppressor protein p53 by way of the UPS.

In the development of colorectal cancer, β-catenin, a cellular factor which is regulated by way of the UPS, plays an important role in the signal transduction and differentiation of colorectal epithelium. In addition, a correlation exists between the level of p27, a G1 cyclin CDK inhibitor, and the development of colorectal cancer and breast cancer. The breakdown of p27 by the UPS is crucial for the transition from the G1 phase to the S phase during cell division.

The UPS is known to play a role in hereditary diseases, for example in the pathomechanisms of cystic fibrosis, Angelman syndrome and Liddle's syndrome. UPS plays a role in neurodegenerative diseases as well: the accumulation of ubiquitin conjugates in pathological lesions has been reported in the case of Alzheimer's disease and in the case of Parkinson's disease. In Huntington's disease, the proteins huntingtin and ataxin accumulate in proteasome-active nuclear structures in the cell nucleus. The UPS exercises a central function in diseases of the immune system. In the first place, the 26S proteasome complex is the main protease in MHC I antigen processing and, in the second place, the activity of proteasome itself can be manipulated both by γ-interferon-inducible catalytic β subunits and by the regulatory subunit PA28. Many inflammatory and immunological diseases are connected with the transcription factor NF-κB, which regulates a variety of gene functions in the immune response. The activation of NF-κB, which is controlled by ubiquitinylation and the specific cleavage of a precursor protein by the proteasome, leads to an increase in the expression of a variety of cytokines, adhesion molecules, inflammatory and stress-response proteins and immune receptors.

These correlations explain the interest in a pharmacological application of substances which regulate the UPS.

1.2. Proteasome Inhibitors

Various substance classes are known to be proteasome inhibitors. On the one hand, they are chemically modified peptide aldehydes such as the tripeptide aldehyde N-carbobenzoxyl-L-leucinyl-L-leucinyl-L-leucinal (zLLL: also designated MG132) and the more active boric acid derivative MG232.zLLL and derivatives thereof. They block the proteasome reversibly by forming a transient hemiacetal structure with the catalytically active threonine hydroxyl side chain in position 1 of the β subunit of the 26S proteasome. Like zLLL, another class of modified peptides, i.e. the peptide vinylsulfones, have been reported to be proteasome inhibitors (for a review, see Elliott and Ross, 2001).

Lactacystin (LC) (Fenteany et al., 1995), which is obtained from streptomycetes, and epoxomycin, which is obtained from actinomycetes (Meng et al., 1999a,b), are naturally occurring substances. LC is a highly specific proteasome inhibitor which acts irreversibly and which principally blocks the chymotrypsin and trypsin-like activities of the 26S proteasome particle (Fenteany et al., 1995). LC does not have any basic peptide structure but instead consists of a γ-lactam ring, a cysteine and a hydroxybutyl group. LC does not itself inhibit the proteasome. Instead, the N-acetylcysteine radical is hydrolyzed in aqueous solution. This results in the formation of a clastolactacysteine β-lactone which is able to penetrate cell membranes. Following uptake into the cell, the β-lactone ring performs a nucleophilic attack and the threoninel hydroxyl group of the β subunit is then transesterified (Fenteany et al., 1995). Epoxomycin is thus far the most effective, as regards specificity and activity, of all the known natural proteasome inhibitors (Meng et al., 1999; a,b).

Another, and very potent, class of synthetic proteasome inhibitors are boric acid peptide derivatives, in particular the compound pyranosyl-phenyl-leucinyl-boric acid, which is named “PS-341”. PS-341 is very stable under physiological conditions and is bioavailable following intravenous administration (Adams and Stein, 1996; Adams et al., 1996, US 1448.012TW01). The particular efficacy of PS-341 as a proteasome inhibitor is probably due to the very stable bond between the boric acid group and hydroxyl group of the catalytically active side chain of Thr1 in the active β subunit of the 20S proteasome (Ki=0.6 nM) (Adams and Stein, 1996). PS-341 is thus far not known to influence any cellular protease other than the proteasome. Various boric acid-peptide derivatives have already been tested for their effect as proteasome inhibitors (Adams et al., 1998). In this connection, it has been found that leucine, preferably in the P1 position, and relatively large hydrophobic side chains (for example naphthylalanine) in P2 and P3 improve the efficacy and the Ki value (inhibitory constant) of the inhibitor (Adams et al., 1998).

1.2.1. Clinical Application of Proteasome Inhibitors

Inhibition of proteasome activity, as the main cellular protease, can lead to changes in the regulation of the cell cycle, of transcription, of overall cellular proteolysis and of MHC-1 antigen processing (for a review, see Ciechanover et al., 2000). Consequently, long-lasting inhibition of all the enzymic activities of the proteasome is not compatible with the survival of a cell and consequently of the whole organism. However, certain reversibly acting proteasome inhibitors can selectively inhibit individual proteolytic activities of the 26S proteasome without at the same time having any effect on other cellular proteases. The cytotoxicity of these inhibitors is therefore substantially less than that of peptide aldehydes, such as zLLL, which act in a relatively nonspecific manner. The first clinical studies carried out using proteasome inhibitors (Adams et al., 1999) make it clear that this class of substances has an enormous potential for use as pharmacological agents having a diverse application basis (for a review, see Elliot and Ross, 2001).

The significance of proteasome inhibitors as a novel therapeutic principle has experienced increased attention in recent years in particular in connection with treating cancer and inflammatory diseases (for a review, see Elliot and Ross, 2001). While their broad clinical use in humans has not yet been authorized, the pharmaceutical industry is working intensively on developing new medicaments which are based on proteasome inhibitors which are tolerated in vivo. The company “Millennium Inc.” (Cambridge, Mass., USA) has developed proteasome inhibitors, in particular boric acid derivatives of dipeptides and, in this connection, particularly the compound PS-341 (Adams et al., 1999), for antiinflammatory, immunomodulatory and antineo-plastic therapies. In the rat model, the oral administration of PS-341 has an inflammation-inhibiting effect in streptococcus-induced rheumatoid arthritis and liver inflammation (Palombella et al., 1998). In the mouse model, PS-341 exhibits an antineoplastic effect against lung carcinomas and, in addition to this, has an additive effect in combination with cytostatic agents (Teicher et al., 1999). In vitro experiments demonstrate that the compound has very good activity against solid human ovarian and prostate tumor cells (Frankel et al., 2000). PS-341 is to date the only proteasome inhibitor to have been subjected to clinical trials. Phase I clinical studies on PS-341 demonstrate good bioavailability and pharmacokinetic behavior (Lightcap et al., 2000). Phase I and phase II clinical studies in patients suffering from various cancer diseases, such as hematological malignancies as solid tumors, have already been concluded. In addition to surprising therapeutic effects being achieved in different tumor patients, it is noteworthy that it was not possible to observe any dose-limiting toxicity when treating with PS-341. Millennium Inc. has presented the information in this regard in communications to the press (published under
http:biz.yahoo.com/prnews/010301/neth003.html;
http:biz.yahoo.com/prnews/010301/neth003.html;
http://www.mlnm.com.releases.pr052300_l.shtml;
http://www.cancernet.nci.nij.gov/;
http://www3.manderson.org/leukemia/insight/letter52.html

Another clinical application of proteasome inhibitors, particularly those developed by Millennium Inc., is indicated in connection with inflammatory diseases and autoimmune diseases. These diseases are in general elicited by a cascade of cytokine and chemokine production and by the expression of particular cell adhesion molecules. The transcription factor NF-κB occupies a central position in this context. NF-κB is required for expressing a number of proinflammatory factors. As a representative of the Rel proteins, it consists of a heterodimer composed of p50 and p65 (RelA) subunits (for a review, see Baldwin, 1996). In resting cells, the binding of the inhibitory factor IκB to NF-κB guarantees, by masking the nuclear localization signal of the p50/p65 heterodimer, localization of NF-κB in the cytosol in a latent, inactive form. Cell activation signals, for example cytokines or viral infections, trigger the phosphorylation and polyubiquitinylation of IκB and consequently the activation of NF-κB. Following activation, NF-κB translocates into the cell nucleus and stimulates the transcription of a variety of genes, especially those for cytokines, chemokines and cell adhesion molecules. These factors are in sum involved in regulating immunological and inflammatory processes. Proteasome inhibitors are able to block the breakdown of IκB and consequently the activation of NF-κB (Palombella et al., 1994).

It has been shown in a mouse model that the proteasome inhibitor PS-519 (a β-lactone derivative) exerts a powerful antiinflammatory effect. In low doses, PS-519 is also effective in combination with steroids. PS-519 has therefore been proposed as a novel drug for treating asthma (Elliot et al., 1999). Another application for PS-519 is seen in the infarction model: PS-519 dramatically reduced the inflammatory reaction following cerebral injury. Accordingly, PS-519 also appears to be a pharmacological agent of interest for treating cerebral stroke (Phillips et al., 2000).

Since proteasome inhibitors act on an essential pathway in cell metabolism, a strict dose regime is required in order to suppress toxic side effects. A variety of peptide-boric acid derivatives exhibiting an antineoplastic effect both in cell culture and in an animal model have been tested within the context of developing proteasome inhibitors which are tolerated in vivo (Adams et al., 1996; 1998; 1999). In vitro, PS-341 exhibits selective cytotoxic activity against a broad spectrum of human tumor cell lines (Adams et al., 1999). This activity is associated with the accumulation of p21 and cell cycle arrest in the G2-M phase with subsequent apoptosis (Adams et al., 1999). In a mouse model, direct injection of PS-341 resulted in the death of 70% of the tumors investigated. In human xenograft models, PS-341 was distributed to all organs and tissues after having been administered intravenously and exhibited antineoplastic activity (Adams et al., 1999). Toxicological studies on PS-341 in primates resulted in dose-dependent side effects, especially in the gastrointestinal region, where PS-341 showed the highest distribution after having been administered intravenously (Adams et al., 1999). Another disadvantage of PS-341 and related inhibitors is that they are unable to overcome the blood/brain barrier and can consequently not become active in the central nervous system (Adams et al., 1999).

The use of proteasome inhibitors with the aim of blocking viral infections has already been described. In particular, Schubert et al. (2000 a, b) demonstrated that proteasome inhibitors block the assembly, release and proteolytic maturation of HIV-1 and HIV-2. This effect is based on a specific blockade of proteolytic processing of the Gag polyproteins by the HIV protease, without proteasome inhibitors having any effect on the enzymic activity of the viral protease itself. The mechanism by which proteasomes regulate the assembly of different viruses is not yet understood. Further connections with the UPS have been reported in regard to the budding of Rous sarcoma virus, RSV (Patnaik et al., 2000); simian immunodeficiency virus, SIV (Strack et al., 2000) and Ebola virus (Harty et al., 2000). In the latter case (Harty et al., 2000), it was shown that a cellular ubiquitin ligase interacts with Ebola matrix protein; thus far, there has been no demonstration of proteasome activity being of direct importance for the replication of Ebola or other related viruses and the inhibition of Ebola virus assembly.

An important part of the invention consequently consists in the fact that the surprisingly observed antiviral effect of proteasome inhibitors on members of the Flaviviridae family, especially on viruses, such as pestiviruses, which are closely related to HCV, has not previously been recorded. In particular, there has not previously been any report of antiviral effects which are due to proteasome inhibitors and which relate to the entry/internalization process, or the uncoating (release of the virus core), of the Flaviviridae virus particle. Nor has there been any report of antiviral effects which are caused by proteasome inhibitors and which relate to the assembly, maturation or secretion of Flaviviridae progeny viruses. There has been no report that proteasome inhibitors block the release of infectious Flaviviridae virions from virus-producing cells. In addition, there has been no report that proteasome inhibitors kill hepatoma cells in preference to primary hepatocytes and are therefore suitable for treating liver carcinomas. The effects, which are described in accordance with the invention, of proteasome inhibitors on early and/or late processes in the Flaviviridae life cycle, and also on hepatoma cells, consequently represent completely novel principles in the antiviral treatment of infections due to these viruses, especially HCV infections.

1.3. Link Between the UPS and the Life Cycle of Flaviviridae, Especially HCV

There has not previously been any analysis, in any known study, of the influence which proteasome inhibitors have on the release and infectivity of members of the Flaviviridae family, as observed and detected in the present invention description. It has previously been published that the expression of HCV proteins does not have any effect on the activity of the UPS (Moradpour et al., 2001). In addition to this, it is known that a fraction of the HCV core protein is broken down by way of the UPS (Suzuki et al., 2001). While a monoubiquitinated form of the core protein also arises, it is stable and is not broken down. It has not previously been tested whether proteasome inhibitors have any influence on the proliferation or transformation status of hepatocellular carcinomas (HCCs). It is only known that the P28 proteasome subunit is overexpressed in most HCCs (Higashitsuji et al., 2000).

2. Biology of the Flaviviridae

The Flaviviridae family encompasses the genera Flavivirus, Pestivirus and Hepacivirus (hepatitis C virus, HCV). In addition to these three genera, another group of Flaviviridae-like viruses, i.e. the GB viruses, has recently been characterized but not yet classified. The phylogenetic relatedness between pestiviruses and HCV is considerably more pronounced than it is between flaviviruses and pestiviruses or between HCV and flaviviruses. Accordingly, the morphology of the virion, the genomic organization, and the known biochemical mechanisms of gene expression and genome replication, are particularly well conserved between pestiviruses and HCV. As has already been explained, many members of the Flaviviridae are important human and animal pathogens (for a review, see Lindenbach and Rice, 2001).

2.1. Construction of the Virion: Mechanism of Entry of the Virus into the Host Cell

The viral genome in Flaviviridae is a single-stranded, unsegmented RNA which is of positive orientation and reaches 10, 11 and 13 kilobases in length in HCV, flaviviruses and pestiviruses, respectively. In the virion, the RNA genome is complexed with capsid (C, or core) proteins. This nucleoprotein complex is surrounded by a lipid double membrane in which two or three species of envelope (E) proteins are embedded. It is assumed that binding of the virus particle to the cell, and entry, take place by way of receptor-mediated endocytosis. In this process, the viral envelope proteins associate with a cellular receptor: in the case of HCV, various experimental data point to the envelope protein E2 interacting with CD81 and/or the low density lipoprotein (LDL) receptor (for a review, see Lindenbach and Rice, 2001). In the case of BVDV, it has been found that there is an interaction with CD 46 and the LDL receptor (Müller et al. 2000; Agnello et al., 2000). As a result of the virus envelope fusing with the membrane, with this being catalyzed by a low pH, the nucleocapsid is released into the cytoplasm. An unknown mechanism leads to dissociation of the viral capsid proteins and release (uncoating) of the genome (for a review, see Lindenbach and Rice, 2001).

2.2. Gene Expression in the Flaviviridae

The viral RNA comprises a single translational reading frame (open reading frame; ORF) which is flanked at the 5′ and 3′ ends by untranslated regions (UTRs). Translation leads to the synthesis of an unstable polyprotein which is cleaved cotranslationally and posttranslationally into viral polypeptides.

In conformity with the similarities in the genome structure, the events which lead to the processing of the polyprotein are to a large extent conserved in the members of the Flaviviridae. This applies, in particular, to pestiviruses and HCV. The aminoterminal region of the polyprotein contains the polypeptide sequences for the structural components of the virion, i.e. for the capsid protein, C, and for at least two viral envelope proteins (flaviviruses: prM, E, NS1; pestiviruses: E^{ms}, E1, E2; HCV: E1, E2). The structural proteins are primarily generated from the polyprotein by host cell signal peptidases. The envelope proteins, which are strongly glycosylated, associate to give rise to homodimers and heterodimers: E2-E2 and E2-E1 dimers have been reported in pestiviruses and HCV. The function of the soluble pestiviral envelope protein E^{ms} is thus far unclear: it possesses a ribonuclease function and is not required for infection in cell culture. The dimerization plays an important role in connection with virus entry (for a review, see Lindenbach and Rice, 2001). In all three genera, the processing of the C-terminal nonstructural (NS) polyprotein NS2A-NS2B-NS3—NS4A-NS4B-NS5 (in the case of flaviviruses) or NS2—NS3 (in the case of pestiviruses NS2-3)-NS4A-NS4B-NS5A-NS5B (in the case of pestiviruses and HCV) is mainly catalyzed by a viral protease complex. This complex consists of a serine protease, which is located in the amino terminal region of the NS3 protein, and a cofactor (NS2B in the case of flaviviruses and NS4A in the case of pestiviruses and HCV). In flaviviruses, the NS3—NS2B complex generates all of the NS proteins; only the proteolysis between NS4A-NS4B is catalyzed by a cellular signalase activity. In pestiviruses and HCV, NS3-4A cleaves the complete NS polyprotein with the exception of NS2—NS3. In HCV, the latter cleavage is catalyzed by an autoprotease which is located at this site (for a review, see Lindenbach and Rice, 2001). In the pestivirus BVDV, the extent of the proteolytic cleavage between NS2 and NS3 is interestingly linked to a cytopathic effect of the viral infection.

It has furthermore been shown that NS5 (in the case of flaviruses) and NS5B (pestiviruses, HCV) is the viral RNA-dependent RNA polymerase (RdRp). The functions of NS4B and NS5A have not thus far been clarified.

2.3. Gemone Replication in the Flaviviridae

In accordance with the general replication model for positive-strand RNA viruses, a functional membrane-bound replication complex, which consists of the viral RNA and viral and cellular factors, associates in the cytoplasm of the Flaviviridae-infected host cell in direct functional coupling to translation and proteolysis of the polyprotein. This complex initially catalyzes the synthesis of a small number of complementary minus-strand RNA molecules; these then serve, in a second step, as the template for synthesizing a significant excess of new plus-strand RNA genomes.

The RNA replication process has been intensively investigated in recent years, particularly in pestiviruses. Important reasons for this were the lack, which has already been mentioned, of an infectable cell culture system for HCV and the fact that pestiviruses act as a model for HCV. Based on data obtained with BVDV, it has also recently been possible to establish an efficient system for replicating HCV (Lohmann et al. 1999). Infectable cultured cells have been available for pestiviruses for some considerable time. Furthermore, “infectious cDNA constructs”, from which it is possible to obtain complete viral RNA genome molecules by in vitro transcription, were constructed for BVDV and CSFV in 1996. After having been transfected into host cells, these RNA molecules are able to pass through the complete pestiviral life cycle up to the synthesis of new virus particles (Moormann et al., 1996; Meyers et al., 1996). By mutagenizing the cDNA, it is possible to introduce mutations selectively into the viral RNA transcripts and determine their effect on the viral life cycle (“reverse genetics”).

The identical organization of pestivirus and HCV replicons is an unambiguous indication that the general molecular mechanisms which underlie viral RNA replication are conserved between the two genera. However, in the case of HCV, it has not yet been possible to reproduce the viral life cycle in cell culture either using virus isolates from patients or using complete genomic RNA which has been transcribed in vitro (Pietschmann et al., 2002). Consequently, it is necessary to rely on the pestivirus model when investigating entry of the virus into the cell, uncoating of the genome and maturation and secretion of progeny viruses.

2.4. Virion Assembly and Secretion of Progeny Viruses

The late steps in the viral life cycle have hardly been investigated in any of the three Flaviviridae genera. It is assumed that a fraction of the newly synthesized positive-strand RNA molecules associates in “statu nascendi” with capsid proteins to form nucleocapsid particles. As a result of budding, these particles come to be located in cytoplasmic vesicles belonging to the endoplasmic reticulum. Since, during maturation, the envelope proteins remain in the ER membrane, this thereby gives rise to the coated virions, which contain the envelope proteins in the correct orientation. During the course of cellular secretion, the vesicles fuse with the plasma membrane, resulting in the virus particles being released into the extracellular compartment (Lindenbach and Rice, 2001).

2.5. The Problem of Heterogeneity

Mutations frequently arise during the replication of Flaviviridae. For this reason, the corresponding host organisms are always infected with a very heterogeneous population of these viruses. The heterogeneity affects pathogenesis, resistance to the virus, the response to therapy with interferons (IFNs) and antiviral substances (nucleoside analogs and others) and the recognition of infected cells by the immune system. This fact provides support for novel antiviral strategies being necessary and appropriate.

2.6. Options for Treating a Chronic HCV Infection

One of the few options for treating a chronic HCV infection is that of treating it with interferon (IFN) alpha and derivatives. An important disadvantage of IFN therapies is that they are frequently associated with negative side effects. Since 1999, the use of the guanosine analog ribavirin in combination with interferons has been approved for treating chronic HCV. However, the mode of action of this medicament has not been completely clarified. In addition, ribavirin has a number of side effects, with ribavirin-induced hemolysis being of particular importance. Other nucleoside analogs and a number of substances whose mode of action is unknown are used in addition to ribavirin (for a review, see Trautwein and Manns, 2001). With all the medicaments which are at present authorized for treating an HCV infection, there are a large number of nonresponders (as a rule about 30-40%). The success rate is even lower when various viruses are coinfecting (e.g. human immunodeficiency virus HIV and HCV or HBV and HCV). Even when the treatment is clinically successful, all the viral reservoirs are only rarely completely eliminated and these reservoirs can therefore lead to the infection being reactivated (Rehermann et al., 1996). When there is a lack of response to the therapy, or even reactivation of the virus replication, it is in principle only novel medicaments, such as those described in this invention, which can then help. It is not yet possible to assess with confidence the risks of using all the antiviral medicaments which have been employed thus far. In principle, nucleoside analogs such as ribavirin carry the risk of resistant viruses developing and of mutations taking place in the host genome, with these mutations possibly giving cause for the development of cancer. The novel medicaments which are mentioned within the context of the invention which is presented here are either not associated with these risks, or else these risks are much less probable or not to be expected.

The following patent specifications, which do not directly affect the present invention, have been published: a method for determining proteasome activity in biological samples (WO 00/23614); the use of proteasome inhibitors as agents for treating cancer, inflammation and autoimmune diseases (WO 99/22729); the use of inhibitors of the UPS as agents for treating inflammations and autoimmune diseases (WO 99/15183).

Nature of the Invention

The invention is based on the object of making available agents for inhibiting the release and infectivity of members of the Flaviviridae, in particular hepatitis C viruses. The object was achieved by using at least one proteasome inhibitor in a pharmaceutical preparation. According to a preferred embodiment of the invention, substances which inhibit, regulate or otherwise affect the activities of the cellular proteasome pathway can be used as proteasome inhibitors. It is also possible to make use, as proteasome inhibitors, of substances which specifically affect the enzymic activities of the complete 26S proteasome complex and of the free 20S, catalytically active, proteasome structure, which is not assembled with regulatory subunits. These inhibitors can inhibit either one or more, or all, of the three main proteolytic activities of the proteasome (the trypsin, chymotrypsin and postglutamyl peptide-hydrolyzing activities) within the 26S or the 20S proteasome complex.

A variant of the invention consists in making use, as proteasome inhibitors, of substances which are taken up by higher eukaryotic cells and, after having been taken up into a cell, interact with the catalytic beta subunit of the 26S proteasome and, in connection with this, irreversibly or reversibly block all or some of the proteolytic activities of the proteasome complex.

As another form of the invention, use is made of agents which inhibit the activities of the ubiquitin-conjugating and ubiquitin-hydrolyzing enzymes. In a general manner, polyubiquitination is regarded as being a recognition signal for proteolysis by the 26S proteasome, and an effect on the ubiquitin pathway can also regulate the activity of the proteasome.

According to the invention, use is also made, as proteasome inhibitors, of substances which are administered in various forms in vivo, i.e. orally, intravenously, intramuscularly, subcutaneously, in encapsulated form, with or without cell specificity-carrying changes, or in some other way, which, due to using a particular administration and dose regime, exhibit low cytotoxicity and/or high selectivity for particular cells and organs, which do not elicit any side effects, or only elicit insignificant side effects, and which exhibit a relatively high metabolic half life and a relatively low clearance rate in the body.

Use is furthermore made, as proteasome inhibitors, of substances which are isolated in natural form from microorganisms or other natural sources, which are formed from natural substances as a result of chemical modifications, or are prepared completely synthetically. These substances include:
a) naturally occurring proteasome inhibitors:
   epoxomicin and eponemycin,
   aclacinomycin A (also termed aclarubicin),
   lactacystin and its chemically modified variants, in particular the cell membrane-penetrating variant “clastolactacystein β-lactone”,
b) synthetically prepared:
   modified peptide aldehydes, such as N-carbobenzoxy-L-leucinyl-L-leucinyl-L-leucinal (also designated as MG132 or zLLL), its boric acid derivative MG232; N-carbobenzoxy-Leu-Leu-Nva-H (designated MG115); N-acetyl-L-leucinyl-L-leucinyl-L-norleucinal (designated LLnL); N-carbobenzoxy-Ile-Glu(OBut)-Ala-Leu-H (also designated PSI);
   peptides which carry α,β-epoxyketones (also termed epoxomicin or eponemycin), vinylsulfones (for example carbobenzoxy-L-leucinyl-L-leucinyl-L-leucinevinylsulfone, or 4-hydroxy-5-iodo-3-nitrophenylacetyl-L-leucinyl-L-leucinyl-L-leucine-vinylsulfone, also termed NLVS), glyoxal or boric acid radicals (for example pyrazyl-CONH(CHPhe)CONH(CHisobutyl)B(OH)₂), also termed “PS-431” or benzoyl (Bz)-Phe-boroLeu, phenacetyl-Leu-Leu-boroLeu, Cbz-Phe-boroLeu); pinacol esters, for example benzyloxycarbonyl (Cbz)-Leu-Leu-boroLeu-pinacol ester, C-terminally; and
   use is made of peptide and peptide derivatives, as particularly suitable compounds, which carry epoxyketone structures C-terminally; these include, for example, epoxomicin (molecular formula: C₂₈H₈₆N₄O₇) and eponemycin (molecular formula: C₂₀H₃₆N₂O₅);
   chemically modified derivatives based on naturally occurring [lacuna], in particular a β-lactone derivative having the designation PS-519 (IR-[1S,4R,5S]]-1-1-(1-hydroxy-2-methylpropyl)-4-propyl-6-oxa-2-azabicyclo[3.2.0]-heptane-3,7-dione, molecular formula: C₁₂HLgNO₄), which is derived from the natural proteasome inhibitor lactacystin;
   certain dipeptidyl-boric acid derivatives, in particular compounds which are derived from the pyranocyl-phenyl-leucinyl-boric acid derivative having the name “PS-341” (N-pyrazinecarbonyl-L-phenylalanine-L-leucine-boric acid, molecular formula: C₁₉H₂₅BN₄O₄). These derivatives furthermore include the compounds “PS-273” (morpholine-CONH—(CH-naphthyl)-CONH—(CH-isobutyl)-B(OH)₂) and its enantiomer PS-293, the compound PS-296 (8-quinolylsulfonyl-CONH—(CH-naphthyl)-CONH(—CH-isobutyl)-B(OH)₂); the compound PS-303 (NH₂(CH-naphthyl)-CONH—(CH-isobutyl)-B(OH)₂); the compound PS-321 (morpholine-CONH—(CH-naphthyl)-CONH—(CH-phenylalanine)-B(OH)₂); the compound PS-334 (CH₃—NH—(CH-naphthyl-CONH—(CH-isobutyl)-B(OH)₂); the compound PS-325 (2-quinol-CONH—(CH-homophenylalanine)-CONH—(CH-isobutyl)-B(OH)₂); the compound PS-352 (phenylalanine-CH2—CH2-CONH—(CH-phenylalanine)-CONH—(CH-isobutyl)1-B(OH)₂); the compound PS-383 (pyridyl-CONH—(CHpF-phenylalanine)-CONH—(CH-isobutyl)-B(OH)₂. All these compounds have already been described, inter alia in Adams et al. (1999).

In addition to epoxomicin and eponemycin, the proteasome inhibitors PS-519, PS-341 and PS-273 (Millennium Pharmaceuticals Inc., Cambridge, Mass. 02139, USA) have proved to be particularly suitable compounds. These proteasome inhibitors are very potent and very specific for the proteasome and do not block any other cellular proteases and therefore have virtually no side effects. In addition, the proteasome inhibitors PS-341 and PS-519 have been tested both in animal models, for preclinical studies, and in humans (cancer patients), for clinical studies.

The agents which are employed in accordance with the invention are characterized by the fact that they are suitable for inhibiting the release, maturation and replication of hepatitis C virus (HCV) and for the treatment and prophylaxis of HCV-induced hepatitides, flavivirus-induced fever, hemorrhages, leukopenia, thrombocytopenia, diarrheal diseases, encephalitides and also pestivirus-induced diseases.

Within the context of the present invention, it is found, surprisingly, that proteasome inhibitors inhibit early or late processes in the life cycle of the BVDV pestivirus and the West Nile flavivirus. By contrast, the proteasome inhibitors have no effect, or hardly have any effect, on the intracellular replication of BVDV and HCV RNA replicons. Specifically, it has been observed that the use, according to the invention, of proteasome inhibitors is suitable for preventing, to a large extent or completely, the production of infectious virions in BVDV-infected and West Nile-infected cells. After BVDV-producing and West Nile virus-producing cells have been treated with proteasome inhibitor, there is a virtually complete reduction in the infectivity of the virions which are released. As a consequence of these novel activities, it is possible to use proteasome inhibitors to suppress the fresh infection of host cells, and thus the spread of an infection in vivo, in the case of the various members of the Flaviviridae family.

As another important item in the invention decription, which item is of importance especially in connection with HCV infections, it has been found that treatment of hepatoma cells with proteasome inhibitors preferentially induces the death of these cancer cells whereas healthy, primary hepatocytes and other nonproliferating liver cells are very much more resistant to a treatment with proteasome inhibitors. It is scarcely possible to treat liver carcinomas medicinally and, without liver transplantation or liver resection, these carcinomas as a rule lead to death. This provides proteasome inhibitors with another therapeutic potential for treating infections with hepatitis C virus: treating with proteasome inhibitors can not only suppress or prevent the spread of the infection (by blocking the production of infectious virions) but also suppress or prevent the development, which is associated with the infection, of liver cell carcinomas, or heal a liver cell carcinoma which is already established. This claim is based on the fact that, in a similar way to the already known antineoplastic effect of proteasome inhibitors on a large number of tumors, treatment with proteasome inhibitors can bring about a specific elimination of liver carcinoma cells in vivo. The antineoplastic effect of proteasome inhibitors has not previously been demonstrated in the case of liver cell carcinomas and therefore constitutes a novel therapeutic principle. Proteasome inhibitors can consequently be used for treating/controlling/preventing HCV-induced liver cirrhosis, in particular primary liver cell carcinomas.

Because infections with hepatitis C are widely distributed and of particularly pathogenicity, and because chronic infections are associated with the development of a liver carcinoma, treatment of these infections with proteasome inhibitors is of particular importance. The data obtained with BVDV and West Nile virus furthermore justify the claim that proteasome inhibitors can also be employed, using the novel antiviral effect, for treating other hepatitis viruses (A, B, D, E and F), GB viruses and other Flaviviridae such as dengue fever virus or early summer meningoencephalitis (FSME) virus.

The proteasome inhibitors can also be used in combination with other anti-hepatitis medicaments and other therapeutic schemes, for example interferon alpha/beta/gamma and variants thereof (for example pegylated interferons), interleukins, nucleoside analogs (lamivudine, cidovir, ribavirin and others), steroids, plasma exchange, thymosin alpha 1, vaccines, passive and active vaccination, therapeutic and prophylactic vaccination, glycyrrhizin, stem cell transplantation, organ transplantations, dietary therapy, immunosuppressive agents, cyclosporins and derivatives thereof, amanditine and derivatives, interleukins and other cytokines, non-proteasome-selective protease inhibitors, azathioprine, hemodialysis and also highly active antiretroviral therapy (“HAART”) in the case of coinfections with HCV and HIV. Since proteasome inhibitors also exert an antiviral effect on HIV, treatment of HCV/HIV coinfections, particularly in combination with HAART therapy, is one of the main areas for applying the invention.

The use, according to the invention, of proteasome inhibitors consists in inhibiting the entry/internalization process and uncoating process of Flaviviridae and in inhibiting the assembly, maturation and release of progeny viruses. The use for inhibiting the replication of Flaviviridae is effected in accordance with the mechanisms
a) blocking/reducing the assembly and release of new virions
b) blocking/reducing the infectivity of the released virions
c) blocking/reducing the spread of infection in cultured cells.

This implies that proteasome inhibitors inhibit the spread of progeny viruses in infected organs.

Another use of proteasome inhibitors lies in inducing the death of hepatocarcinoma cells, in suppressing and/or preventing the development of liver cell carcinomas and in the treatment of patients who have established liver cell carcinomas.

Another use consists in treating/controlling/preventing
HCV-induced liver cirrhosis and/or
HCV-induced liver cell carcinomas
medicament-induced liver carcinomas
genetically determined carcinomas and/or
environmentally determined liver carcinomas.

Another use lies in selectively eliminating liver cell carcinomas which develop as a result of an
HCV infection and/or
HCV-HBV coinfection
HCV-HBV-HDV coinfections.

In addition, proteasome inhibitors are used for
preventing the development, growth and metastasis of liver cell tumors and for preferentially destroying liver carcinoma cells in HCV-infected patients
modulating the expression, modification and activity of the tumor suppressor protein p53 and other HCC-relevant tumor suppressor proteins
liver cell regenerations in patients suffering from hepatitis
reducing the number of infected virus-producing cells in liver cell tissue
inhibiting both the maintenance and persistence of a previously established infection and of a secondary infection and consequently the spread of an infection, including blocking the spread of an HCV infection in vivo
treating coinfections with HCV and immuno-deficiency viruses HIV-1 and HIV-2-treating HCV/HIV coinfections in combination with HAART therapy
preventing a reinfection with HCV in connection with liver transplantations and other organ transplantations
preventing a reinfection with HCV in connection with cell therapies, by means of administering the agents before, during and after the transplantation
treating and controlling hepatitides in combination with each other
preventing a reinfection with HCV in connection with the transplantation of virus-free organs to chronic virus carriers who possess residual virus and can infect new organs, and also in connection with the transfer of virus-containing organs from donors to virus-free patients
preventing the establishment of a systemic hepatitis virus infection immediately following contact with infectious virus, or for
decreasing or eliminating the hepatitis by means of immune system-mediated mechanisms.

Another use of proteasome inhibitors consists in preventing the establishment of a systemic hepatitis virus infection immediately after contact with infectious virus (for example in the case of needle-prick injuries involving virus-contaminated blood or blood products).

Another use of proteasome inhibitors is that of preventing a hepatitis virus infection in individuals who are at high risk of a fresh infection, for example in the case of doctors and other at risk personnel, drug addicts and travelers in regions which are highly endemic for hepatitis viruses, and in patient treatment and for the members of families of chronic virus carriers.

Another use of proteasome inhibitors consists in preventing a reinfection with HCV in connection with the transplantation of the liver and other organs and in connection with cell therapies, achieved by administering the agents before, during, and some time after the transplantation. The administration of these agents is indicated both for the high-risk situation of transplanting virus-free organs to chronic virus carriers who are still harboring residual virus, and where new organs can be infected, and for transferring virus-containing organs from donors to virus-free patients.

Another use consists in treating HCV-induced autoimmune diseases such as mixed type II cryoglobulinemia.

Another use lies in combining proteasome inhibitors with therapeutic agents which are already used in anti-HCV therapy.

An important use consists in employing proteasome inhibitors for producing agents or pharmaceutical preparations for preventing the release, maturation and replication of hepatitis viruses and also for producing pharmaceuticals for the treatment and prophylaxis of hepatitides.

Another use consists in proteasome inhibitors altering the posttranslational modification of the viral structural proteins and consequently reducing or blocking the release and infectivity of Flaviviridae.

Another use of proteasome inhibitors consists in treating flavivirus-infected individuals, that is, for example, individuals who have fallen acutely ill with West Nile fever, yellow fever, dengue fever (7-day fever or dengue hemorrhagic fever) or arbovirus encephalitis. In this case, too, proteasome inhibitors can be used for preventing infection with West Nile virus, dengue fever virus, yellow fever virus or FSME virus in the case of individuals at risk, such as doctors or travelers in highly endemic regions.

Another example of an application is that of treating pestivirus-infected stabled animals with proteasome inhibitors.

In order to achieve the object, molecular virological, biochemical, immunobiological and electron microscopical studies were carried out, within the context of the invention, on cells which had been infected with various representatives of the Flaviviridae or transfected with viral RNA molecules. In accordance with the invention, the defects induced by the proteasome inhibitors were determined using the following agents and methods: (i) virus preparations of BVDV (strain CP7) and West Nile flavivirus having a defined titer of infectious units; (ii) virus endpoint titration methods achieved by means of microscopically detecting infectious viral particles by means of plaque formation or immunostaining methods; (iii) cDNA constructs for preparing subgenomic BVDV RNA replicons and HCV RNA replicons by means of in vitro transcription; (IV) RNase protection methods for detecting/quantifying viral RNA molecules; (v) immunofluorescence tests for determining the ability of viral RNA molecules to replicate or for determining the spread of an infection; (vi) electron microscopic methods for investigating the morphology of viral particles during and after the process of infection (vii) Western blotting studies and immunoprecipitation methods carried out on BVDV proteins and HCV proteins.

The principle of the achievement of the object is demonstrated taking as examples BVDV and West Nile virus, i.e. two characteristic representatives of the Pestivirus and Flavivirus genera, respectively, of the Flaviviridae family. It was first of all shown, in control experiments, that pretreating the target cells (MDBK cells in the case of BVDV, BHK-21 cells in the case of West Nile virus) with noncytotoxic concentrations of various proteasome inhibitor substance classes did not have any effect on a subsequent infection with the viruses. It was likewise shown that pretreating BVD viruses and West Nile viruses with nontoxic concentrations of different proteasome inhibitors did not have any effect on their infectivity.

Within the context of the invention, it is shown, for the first time, that treating previously infected cells with various proteasome inhibitors significantly inhibits the production of infectious virus particles: by means of carrying out virus end point titration on uninfected cells, it was found, according to the invention, that the number of infectious progeny viruses which are released from infected cells which have been treated with proteasome inhibitors falls by several log steps as compared with mock-treated cells. In addition to the drastic reduction in the number of progeny viruses, treatment with proteasome inhibitors also causes a marked decrease in the specific infectivity of the progeny viruses. The specific infectivity was determined by the number of viral genomes (measured by quantitative RNase protection) which were released into the culture supernatant from infected cells as compared with the titer of the infectious units. In immunofluorescence studies, it became clear that both RNA replication and the spread of the infection in cell culture are greatly reduced under the influence of different proteasome inhibitors. The reduction in RNA replication in the infected and proteasome inhibitor-treated cells was confirmed, according to the invention, by using RNase protection to measure the RNA replication products.

It has furthermore been found, by using Western blotting kinetics studies, that the release of virus from BVDV-infected cells is significantly reduced when these cells have been treated with proteasome inhibitors a short time after an infection.

In surprising contrast to this, it was found, in another part of the invention, that the different proteasome inhibitor substance classes only have slight effects on the replication of BVDV RNA replicons and HCV RNA replicons which are persistently transfected in MBDK or Huh-7 cells, respectively. These results showed that proteasome inhibitors do not interfere, or only interfere to a slight extent, in the following processes catalyzed by viral elements and factors: (i) in the translation of viral RNA by way of the IHRES (internal ribosomal entry site) element contained in the 5′ UTR; (ii) in the proteolysis of the nonstructural polyprotein which is mediated by the viral protease complexes NS3/NS4A and NS2—NS3, respectively; (iii) in the replication of the viral RNA which is catalyzed by the viral proteins NS3, NS4A, NS4B, NS5A, and NS5B (and hypothetical intermediates of the nonstructural polyprotein) and the untranslated regions of the genome. Consequently, the targets of classical antiviral substances, i.e. the IRES, the viral proteases, the NS3 ATPase/RNA helicase and the NS5/NS5B-RdRp (see introduction), are not inhibited, or only inhibited to a trivial extent, by proteasome inhibitors. Accordingly, the drastic decrease in the secretion of infectious progeny viruses which is seen when treating infected cells with proteasome inhibitors cannot be attributed, or cannot be exclusively attributed, to the decrease in the RNA replication rate which can be observed but must consequently relate to processes which affect virus entry and/or the uncoating and/or the assembly and/or secretion of progeny viruses.

This hypothesis was supported, in accordance with the invention, by means of virus/cell adhesion experiments and electron microscopic investigations. Thus, it was found, on the one hand, that progeny viruses which are released from infected cells during proteasome inhibitor treatment adhere significantly less efficiently to target cells. Furthermore, after treating infected cells with proteasome inhibitors, it is possible to observe differences, as compared with mock-treated cells, in the number of virus particles which can be detected generally in the cells by electron microscopy, in the ratio of complete virions to incomplete virions and in the morphology of the secreted progeny viruses.

In another part of the invention, it is shown that, in the presence of proteasome inhibitors, significant changes take place in the posttranslational modification of the BVDV and HCV structural proteins, in the processing of the structural protein polyprotein and in the ability of the envelope proteins to dimerize. These changes could consequently causally explain the effects of proteasome inhibitors on the viral life cycle as observed in pestiviruses and flaviviruses.

It is consequently demonstrated, in accordance with the invention, that the inhibitory effect of proteasome inhibitors on the replication of different representatives of the Flaviviridae family is based on the following mechanisms:
1. Blocking/reducing the viral RNA entry and/or uncoating process.
2. Blocking/reducing the assembly and/or secretion of progeny viruses.
3. Blocking/reducing the infectivity of released virions.
4. Blocking/reducing the spread of the infection.

These mechanisms imply that proteasome inhibitors also block the spread of an infection due to different members of the Flaviviridae family in infected organs. The pronounced homologies between pestiviruses and hepatitis C viruses also justify the assumption that proteasome inhibitors have a similar effect in HCV infections.

In another part of the invention, it is shown that primary liver cells are relatively resistant to proteasome inhibitors (up to a concentration of about 10 μM) whereas proliferating carcinoma cells, including liver carcinoma cells, are already destroyed at 1000-fold lower concentrations of proteasome inhibitors. The preferential death of liver carcinoma cells, as compared with primary hepatocytes, is based in the antineoplastic effect of proteasome inhibitors. Consequently, the fundamental advantage of this invention consists in the fact that treating with proteasome inhibitors can elicit two significant effects which are of great importance for controlling liver diseases caused by HCV infections: in the first place, the treatment inhibits the production of infectious virus particles and thus the spread of the infection in the body. In the second place, the treatment prevents the development, growth and metastasis of liver cell tumors, something which very frequently occurs, after a persistence phase, as the result of an HCV infection. In addition to this, the proteasome inhibitors destroy already existing liver carcinomas but not the normal liver cells, which are either not proliferating or only proliferating to a slight extent.

It is therefore possible, on the basis of this novel treatment method, to use proteasome inhibitors to elicit a wide variety of therapeutic effects in connection with infections due to members of the Flaviviridae family. In addition to the advantages which have been explained above, another important advantage is that this strategy affects cellular factors which are essential for the Flaviviridae life cycle but which have substantially higher genetic stability than do viral factors. Due to the fact that the target structure of this novel antiviral strategy has this genetic stability, the appearance of resistance phenomena, as have been observed in the case of many of the previously known inhibitors of RNA viruses, is not to be expected.

The features of the invention are evident from the elements of the claims and from the description, with both individual features and several features in the form of combinations representing advantageous embodiments for which this document seeks protection. The nature of the invention lies in using known agents for a novel purpose and in a combination of known elements, i.e. the proteasome inhibitors, and a novel effect, i.e. their use for treating Flaviviridae infections, which, in their novel overall effect, give rise to an advantage and the sought-after success which lies in the fact that agents are now available for the prevention, treatment, therapy and inhibition of the diseases which various members of the Flaviviridae virus family (genera: Flavivirus, Pestivirus and Hepacivirus) cause in humans and animals.

In another embodiment of the invention, it is likewise shown that using proteasome inhibitors can prevent the production of BVDV by infected cells. In order to achieve this object, cultured cells are infected with BVDV (strain CP7, NADL) and, after the spread of the infection in the culture during the course of a time kinetics, BVDV virions are isolated and biochemically quantified. This thereby proves that the production of Flaviviridae family virions in an infected cell can be inhibited by using a very wide variety of proteasome inhibitors.

The principle, as explained in the invention description, of using proteasome inhibitors for blocking an infection with Flaviviridae is novel in regard to using a previously known substance class (i.e. the proteasome inhibitors) for a novel activity which can be summarized in the following therapeutic concepts:
blocking the production of infectious Flaviviridae and thereby preventing the spread of an infection in vivo, for example to the liver tissue in an HCV-infected individual;
in connection with the described effects on hepatoma cells, this activity also relates to inducing the death of liver carcinoma cells which have developed as a direct or indirect consequence of an infection with HCV.

At the same time, the use of proteasome inhibitors is also novel in regard to the application principle. There has not previously been any knowledge of substances/principles/methods which predominantly affect early or late processes in the replication of Flaviviridae, especially the release of infectious virions. The fact is furthermore novel that the use of proteasome inhibitors results in the Flaviviridae life cycle being blocked. It is novel that there is substantially less probability of resistance mechanisms developing when proteasome inhibitors are used than when using the previous antiviral methods which have been employed when treating RNA virus infections. This is due to the fact that proteasome inhibitors have less of a direct effect on essential components of the virus and instead act on cellular functions which are evidently important for virion genesis. This can be measured specifically by the effects which proteasome inhibitors have on the modification and processing and association behavior of the viral structural proteins.

The principle of the action of proteasome inhibitors, which, while not preventing initiation of the virus infection, prevent the production of infectious virus particles from cells which are already infected with Flaviviridae, is also novel. This action substantially reduces the quantity of infectious virions (virus load) and consequently the spread of the infection in vivo.

From the sum of these novel mechanisms, it can be stated that, when proteasome inhibitors are used in vivo, the net effect of the decrease in the release of viral particles, which are furthermore not particularly infectious or not infectious at all, should reduce the quantity of infectious virions in an infected organism. The use of proteasome inhibitors on their own or in combination with previously employed antiviral therapies is particularly attractive in the case of HCV.

The invention will be explained in more detail with the aid of implementation examples without being restricted to these examples.

### IMPLEMENTATION EXAMPLES

### Example 1

Primary Tupaia Hepatocytes are Relatively Resistant to the Toxic Effects of Proteasome Inhibitors. By Comparison Proliferating Cells, Including Liver Carcinoma (Hepatoma) Cell Lines, are Substantially More Sensitive to the Toxic Effects of Proteasome Inhibitors.

The UPS is involved in a large number of cellular mechanisms. Accordingly, complete inhibition of proteasome activity over a relatively long period of time is not compatible with the vitality of a cell. However, different cell types exhibit different sensitivities to the toxic effect of the inhibitors. In this connection, it is striking that rapidly dividing and/or activated cells are as a rule more sensitive to proteasome inhibitors than are resting and/or nonactivated cultures. The antineoplastic effect of the proteasome inhibitors is based on this fact. In order to test the toxicity of proteasome inhibitors on various cells lines in culture (MDBK, BHK-21 cells), including transformed human hepatoma cells (Huh-7), dose limiting studies were carried out using proteasome inhibitors. For comparison, the same dose limiting studies using proteasome inhibitors were carried out with primary hepatocytes.

As an example of primary hepatocytes, hepatocytes were obtained from 10-12-week-old Tupaias (Tupaia belangeri), which were maintained and treated in conformity with the international guidelines for experimental animal management. Tupaias were chosen because a recent report suggested that Tupaia hepatocytes could possibly be an in vitro infection model for hepatitis C virus (Zhao et al., 2002). The primary hepatocytes were isolated and grown as described in the protocol of Köck et al. (2001). The Tupaia hepatocytes were plated out on collagen-coated plates (Becton Dickinson Co., Bedford, Mass., USA) at a density of from 2×10⁶ to 3×10⁶ cells/ml of Williams E Medium (2 ml per well). Parallel cultures were treated for 30 hours with increasing doses of given proteasome inhibitors, termed PIs below (in each case 10 μM, 5 μM, 1 μM, 100 nM, 20 nM, 10 nM and 1 nM). About every 12 hours, the morphology and vitality of the cells were examined by light microscopy. In addition, their functionality was determined by means of fluorescence vital staining using fluorescein diacetate (FDA) (Sigma, Deisenhofen, Germany) (Yagi et al., 2001). FDA becomes active and is preferentially taken up by hepatocytes; in the hepatocytes, it is converted, by a lipase which is exclusively expressed in hepatocytes, into fluorescein and causes the cytoplasm to be stained with fluorescence, with this being a sign of the vitality of the hepatocytes and their ability to function. In this experiment, the treated and untreated Tupaia hepatocytes were incubated, at 37° C. for 5 minutes, with FDA-containing Williams E medium (5 μg/ml). These cells were then washed with PBS and the vitality of the hepatocytes was assessed using an inverse epifluorescence microscope. None of the cultures which were treated with up to 1 μM PI exhibited either a morphological change or indications of reduced vitality, i.e. it was not possible to distinguish them from untreated hepatocytes. When the cells were treated with 5 μM PI or higher, marked morphological changes, such as rounded cells and intracellular vacuole formation, were observed in the non-hepatocyte cells which are always present in primary hepatocyte cultures. On the other hand, on the basis of visual assessment and fluorescence intensity, the vitality of the hepat-ocytes was unchanged. It was only at a concentration of 10 μM PI and above that the first signs of a toxic effect also appeared in the hepatocytes. Similar results were observed with different PIs, such as lactacystin and Epoxomicin. In summary, it can be stated that primary Tupaia hepatocytes are able to tolerate relatively high concentrations of up to approx. 10 μM proteasome inhibitor.

In order to test the effect of proteasome inhibitors on proliferating cells and, especially, proliferating hepatoma cells as well, various immortal cell lines of animal and human origin were cultured. As a rule, the cells were cultured in Dulbeccos' modified Eagle's medium (DMEM) (Gibco) which was enriched with the following components: 2% (v/v) amino acid stock solution (0.712 g of L-alanine, 1.2 g of L-aspartic acid, 2.8 g of glycine, 1 g of proline based on ˜800 ml of solution); 0.1 mg of d-biotin/l (Sigma-Aldrich); 25 mg of hypoxanthine/l (Sigma-Aldrich), 3.7 g of NaHCO₃/l (Merck, Darmstadt), 10% (v/v) fetal, heat-inactivated calf serum (Biochrom A G, Berlin); 100 units of penicillin/ml; 100 μg of streptomycin (BioChrom AG, Berlin). On the one hand, tests were carried out using BHK-21 cells and MDBK cells, which were known to be able to be infected with flaviviruses or pestiviruses. Huh-7 cells were used as an example of a hepatoma cell line; these cells are thus far the only line which supports the replication of HCV replicon RNAs (Lohmann et al., 1999. The cells were sown on a 6-well plate at a low density of about 0.5×10⁶. About 24 hours later, the medium was changed and, in parallel assays, treated with different concentrations of various proteasome inhibitors. About 30 hours later, the vitality of the cells was examined either by light microscopy (appraisal of cell morphology) or by means of a trypan blue exclusion test. In the case of these three abovementioned cell lines, it was only at concentrations of ≦20 nM of the different proteasome inhibitors that it was no longer possible to observe any negative effects on the vitality of the cells. This demonstrated that proliferating cells, especially proliferating hepatocytes, are substantially more sensitive to the toxic effect of proteasome inhibitors than are primary hepatacytes. In the examples described below, the different proteasome inhibitors were used at a concentration of 10 nM.

### Example 2

Treatment of Flaviviridae-Infected Cell Cultures with Moderate Concentrations of Proteasome Inhibitors Drastically Reduces the Release and Spread of Infectious Progeny Viruses.

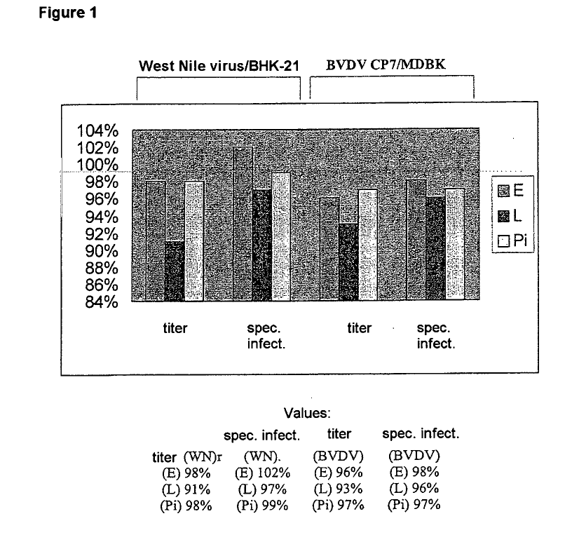

The intention was first to test, in a control experiment, the effect which pretreating the cells with proteasome inhibitors has on an infection with various Flaviviridae members. For this, uninfected MDBK cells and BHK-21 cells were kept, in parallel assays, for different periods of time in cell culture medium to which different PIs (concentration 10 nM) had been added. The time intervals for this treatment ranged from 1 to 8 hours. The cells were then washed with PBS and subsequently treated with a small volume of pestivirus (BVDV CP7)—or flavivirus (West Nile virus)-containing culture medium at an MOI of 1-5 (MOI=multiplicity of infection=number of infectious virus particles used per cell; this had been determined beforehand by virus titration). After an infection phase of about 1 hour at 37° C., excess and/or uninfectious virus was removed by a washing step with PBS and the infected cells were subjected to further culture using fresh culture medium. After one replication cycle, i.e. about 12 hours (West Nile flavivirus) or 30 hours (BVDV), the virus-containing culture supernatant was removed from the cells. Since BVDV CP7 and West Nile virus are cytopathic (cp) viruses, it was as a rule possible to observe, at these times, that the virus infection had had a cytopathic effect (CPE) in the infected cells. The CPE can be identified in the light microscope from the fact that the infected cells are rounded and shrunken and become detached from the culture plate. In addition to this, it was shown, in immunofluorescence tests which were carried out in parallel using specific antibodies directed against viral proteins (only the replication-dependent synthesis of these proteins was measured in this context), that viral replication can be detected in virtually all the cells at these times (standard protocol; see also Behrens et al., 1998). The number of infectious virus particles contained in the culture medium was ascertained by way of an end point titration (standard protocol) on uninfected BHK (West Nile flavivirus) and MDBK (BVDV) cells. Due to the cytopathic character of the two viruses, it was possible to determine the titer microscopically from the formation of plaques in the cell lawn or using a method for immunostaining one of the viral proteins (standard protocols). In order to determine the overall relative number of virus genomes which were present in the cell culture supernatant, a defined volume of the cell culture supernatant was brought to a final concentration of 0.5% (m/v) SDS (sodium dodecylsulfate), 150 mM NaCl and 50 mM tris/Cl, pH 7.5, and the proteins which were present were hydrolyzed by adding 20 μg of the enzyme proteinase K (PK)/ml. The remaining nucleic acids were purified by a phenol extraction and subsequent chloroform extraction and precipitated with ethanol (standard protocols). The viral RNA which was present in the cell supernatant was quantified using a special RNase protection method developed by Behrens et al. (1998) and Grassmann et al. (1999). The general principle of this detection method is based on using short [³²P]-labeled RNA molecules (probes) which are prepared by in vitro transcription (standard protocol) and which exhibit an orientation which is complementary (anti-sense) to that of the viral RNA molecules. After the probes have hybridized to the viral RNA, unpaired regions of the RNA are digested by reaction with the single strand-specific ribonucleases A and T1 and the number of double-stranded (“RNase-protected”) viral RNA molecules is quantified by measuring the labeled probe molecules which are likewise protected (Grassmann et al., 1999). The specific infectivity of the newly assembled progeny viruses was then determined by calculating the ratio of the total number of virus genomes contained in a given volume of cell supernatant and the number of infectious progeny viruses as determined by titration. FIG. 1 shows that pretreating the host cells with different PIs has relatively little effect on a subsequent viral infection: in the case of a flavivirus and pestivirus infection which was carried out after treating the corresponding host cells with proteasome inhibitors, both the titer and the specific infectivity of progeny viruses are either unchanged, or only changed to a slight extent, when compared with a control using mock-treated cells.

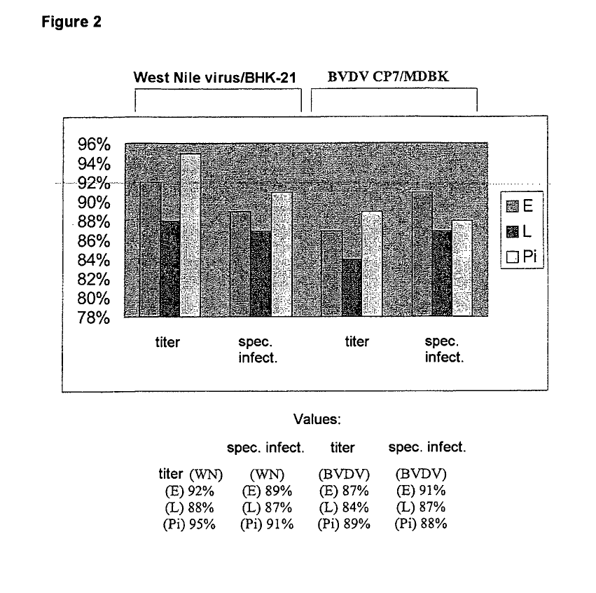

In another control experiment, tests were carried out to determine whether directly treating Flaviviridae virus particles with proteasome inhibitors has any effect on the infectivity of the viruses. For this, culture medium supernatants from infected cells which contained a defined number of West Nile flavivirus or BVDV virus particles (this had previously been determined by virus titration) were treated with different PIs (concentration 10 nM as in the above-described experiments) and incubated at 37° C. for 24 hours. It was found (FIG. 2) that treating the virus supernatant with proteasome inhibitors did not significantly reduce the number of infectious units when compared to an untreated control.

The following can therefore be stated. 1. Treating the virus particles of various representatives of the Flaviviridae with proteasome inhibitors has no effect, or only a slight effect, on the infectivity of these virus particles. 2. Pretreating cells for a maximum of 8 hours with proteasome inhibitors does not have any effect on a subsequent infection of the cells with Flaviviridae; i.e. the proteasome inhibitors do not have any effect, or only have a slight effect, on the cellular structures at the cell membrane which are of importance for virus infection.

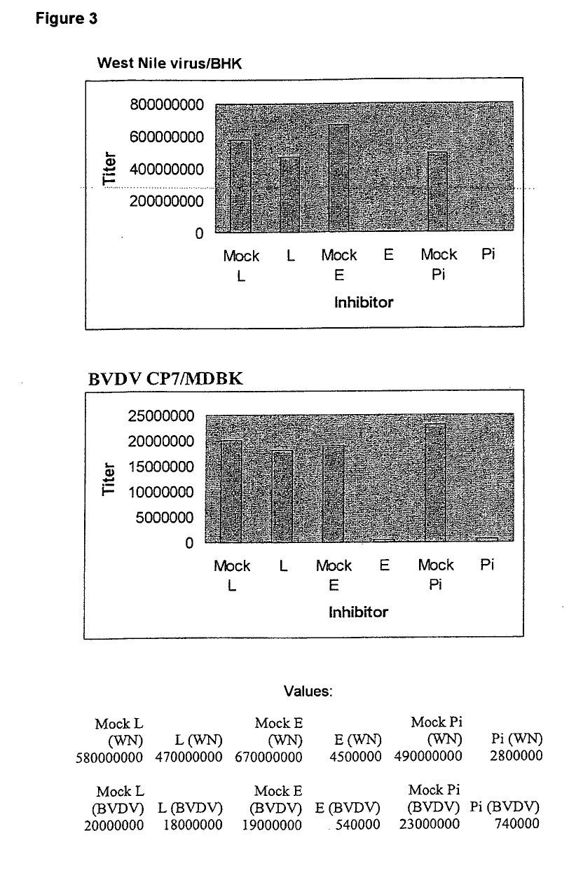

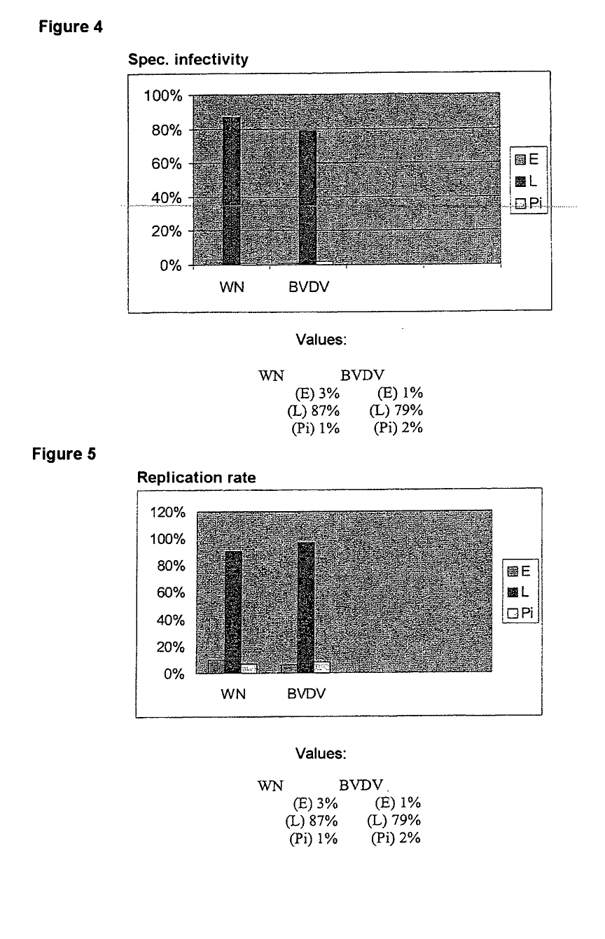

In order to investigate the influence of proteasome inhibitors on the events in a Flaviviridae infection which follow the immediate infection process, such as virus entry/internalization, uncoating, RNA replication and assembly and secretion, and also infectivity of progeny viruses (see introduction), BHK and MDBK cells were infected, in the above mentioned manner, with the West Nile flavivirus and BVDV, respectively, in a series of parallel assays. After the infected cells had been washed with PBS, they were taken up in fresh medium to which 10 nM of different PIs had been added. After 12 hours (West Nil-e flavivirus) or 30 hours of incubation at 37° C., the culture supernatant was obtained in the above-described manner. At the end of the given infection period, the cells were scraped off the culture plate using a rubber policeman, washed in PBS and kept on ice for further use or frozen. The number of newly assembled progeny viruses and their specific infectivity were determined by means of virus titration and said RNase protection method using the culture supernatant. For the purpose of measuring intracellular replication, the viral RNA was isolated from the infected cells. For this, the cells were disrupted, for 10 minutes on ice, in a lysis buffer (50 mM tris/Cl, pH 8, 100 mM NaCl, 5 mM MgCl₂, 0.5% v/v NP40), after which the cell nuclei were separated off by centrifuging at 1000 g and the viral RNA was isolated from the cytoplasm by means of PK digestion, extraction with phenol and chloroform and precipitation with ethanol. The number of viral RNA replication products (Minus-Strang RNA intermediate and newly synthesized Plus-Strang RNA) present in the cells was quantified by RNase protection. The procedure used in order to compensate for the variation in cell number occurring in different infection experiments was as follows: the total quantity of the extracted cytoplasmic RNA was determined by measuring the optical density (OD₂₆₀; standard protocol) (earlier studies showed that only a fraction of the total cytoplasmic RNA is viral RNA even in the case of efficiently replicating viruses) and a defined quantity of this RNA was used in the RNAse protection experiments. FIGS. 3-5 show that both the rate of RNA replication and the number of progeny viruses released were significantly reduced as a result of treating the cells with PI after the infection. However, marked differences were seen between the PIs in this regard; thus, the effect of lactacystin was slight whereas treating with epoxomycin or a proteasome inhibitor, Pi, had a maximum effect, i.e. a reduction in the rate of RNA replication by a factor of about 5-10 and a reduction in the number of progeny viruses released by several log steps. A marked reduction in specific infectivity, i.e. the ratio of the number of RNA genomes which were detectable in the supernatant and the number of infectious units identified in the titration test (by a factor of about 50-100) was also determined.

In order to test whether the lower infectivity of the progeny viruses which was observed is due to a decrease in the ability of the virus particles to adhere to the target cell, the following experiment was carried out: BHK and MDBK cells were treated, in parallel assays, with West Nile flavivirus-containing and, respectively, BVDV-containing supernatants which had been obtained from PI-treated or untreated infection experiments. In this connection, it was important that an MOI which was in each case identical was employed in the infection. After an incubation phase of one hour at 4° C., one part of the cells was brought to 37° C. for one hour while the other part of the cells was immediately subjected to further working-up. The cells were obtained by scraping them from the plate and the total RNA was then isolated from the respective assays by digesting with PK and extracting with phenol and chloroform. The ratio of the viral RNA genomes which adhered to the cells at 4° C. and which remained/were internalized at 37° C. was then quantified by RNAse protection. This experiment showed that the virus particles which had been secreted, as progeny viruses, from proteasome inhibitor-treated cells were substantially less infectious than viruses which had been obtained from untreated cells.

In a consistent manner, it was possible to observe a marked decrease in the spread of the infection in cultures of flavivirus-infected and pestivirus-infected cell lines when proteasome inhibitors were present. This latter fact was particularly impressively verified by means of immunofluorescence tests: whereas typical plaque formations of the infected cells are seen under the microscope in the case of an infection with mock treatment, with these plaque formations being a consequence of the horizontal spread of the virus infection, this was not the case when the cells were treated with PIs (FIG. 5).

In summary, these experiments make it clear that treating infected cells with proteasome inhibitors leads to a blocking/reduction of the replication rate of the viral RNA in infected cells, to a blocking/reduction of the release of new virions and to a blocking/reduction of the infectivity of progeny virus which have been released. As a consequence, the spread of the viral infection is reduced. The observation that proteasome inhibitors exert a considerable inhibitory effect on the life cycle of both flaviviruses and pestiviruses makes it possible to conclude that these agents also have a similar effect on an infection, and the spread of an infection, caused by other members of the Flaviviridae family, such as hepatitis C viruses and GB viruses, for which it has not thus far been possible to establish any infectious cell culture systems.

### Example 3

Proteasome Inhibitors Only Have Slight Effects on the Intracellular Replication of BVDV RNA and HCV RNA.

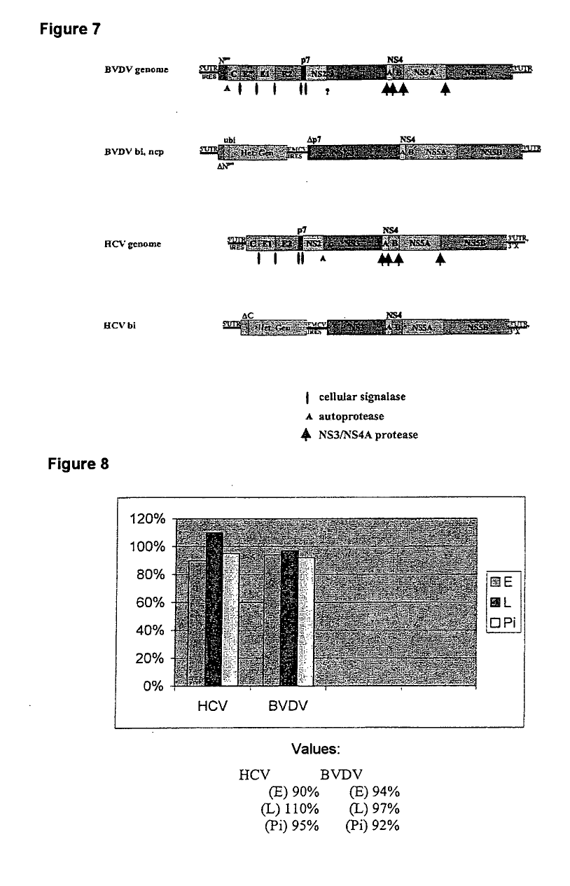

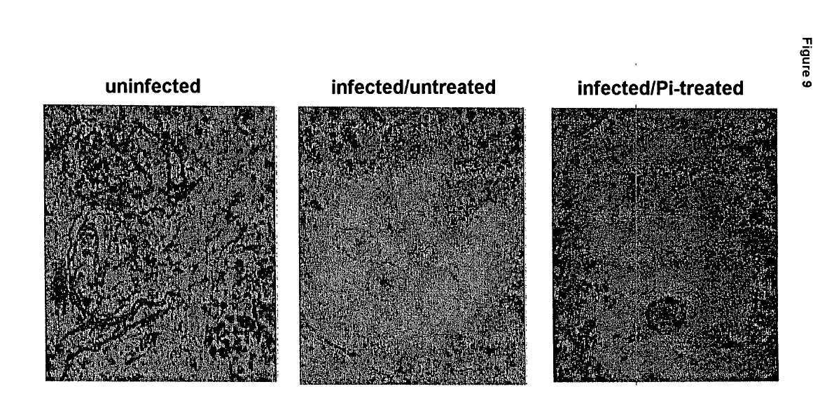

In order to investigate in more detail the effect of proteasome inhibitors on the intracellular replication of the viral RNA genome, recourse was had to cell lines which contain a persistent BVDV RNA replicon or a persistent HCV RNA replicon (FIGS. 7 and 8). In order to prepare these cell lines (MDBK in the case of BVDV and Huh-7 in the case of HCV), use was made of bicistronic RNA replicon constructs which, in addition to the ORF encoding the viral proteins, also contain a further gene which encodes a selection marker, for example the gene for hygromycin B phosphotransferase. Hygromycin B phosphotransferase inactivates the antibiotic hygromycin B, which is an inhibitor of cellular translation. In the bicistronic constructs, the heterologous gene is translated by way of the IRES in the viral 5′-UTR; the viral genes are expressed by way of an encephalomyocarditis virus (EMCV) IRES sequence which has been additionally inserted. In the case of BVDV, a noncytopathic (ncp), NS2-3-encoding replicon was used, in conformity with the natural situation, to prepare a persistently transfected MDBK cell line (see introduction and FIGS. 7 and 8). In the case of HCV, an NS3-expressing RNA was used for preparing persistently transfected Huh-7 cell lines; this RNA does not induce any CPE (Lohmann et al., 1999). After transfecting the RNAs which were obtained by in vitro transcription (standard protocols), the cells were cultured for several weeks under selecting conditions, i.e. in the presence of 500 μg of hygromycin/ml. Since the only cells which survive under these conditions are those which exhibit efficient resistance to hygromycin, this thereby selected homogeneous cell lines which persistently harbor the corresponding “Hyg-bici”-BVDV replicon or “Hyg-bici”-HCV replicon. The advantage of this replicon cell system is, in particular, that effects on RNA replication can be investigated independently of virus entry/internalization and uncoating and the assembly and secretion of progeny viruses. The Hyg-bici-BVDV or Hyg-bici-HCV replicon cell lines were treated with various PIs for from 24 to 30 hours (see below). The effects of the proteasome inhibitors on the intracellular replication of the viral RNAs were in turn determined by measuring the replication products of the viral RNAs using quantitative RNase protection. Alternatively, the effect of the treatment with proteasome inhibitors on viral RNA replication was monitored, as described in Example 2, by way of immunofluorescence tests or using immuno blotting methods (standard protocols). It was found that different PIs either had no effect, or only a slight effect, on the intracellular replication of BVDV RNA and HCV RNA: FIGS. 7 and 8 show that the replication rate of the Hyg-bici-BVDV replicon RNA or Hyg-bici-HCV replicon RNA only decreases slightly (i.e. by a maximum of 10%) during treatment with proteasome inhibitors.

These results show that inhibition of the cellular proteasome does not have any effect, or only a very slight effect, on the translation of the viral RNA, on the processing of the viral nonstructural polyprotein and on the catalysis of the two viral RNA replication steps. Because of the discrepancy in regard to the results obtained with infected cells—as described in Example 2, it was evident that PIs had a clear effect on RNA replication in these cases—it was possible to conclude that proteasome inhibitors evidently inhibit events, such as virus entry/internalization and/or uncoating, which precede replication of the RNA.

### Example 4

The Treatment of Flaviviridae-Infected Cells with Proteasome Inhibitors Leads to Differences in the Number of Virus Particles which can be Detected in Infected Cells, to Changes in the Ratio of Complete and Incomplete Virions and to Changes in the Morphology of Secreted Progeny Viruses.

In the previous experiments (Example 2), it was found that inhibiting cellular proteasome activity drastically reduces the release of infectious progeny viruses. The simultaneous observation, i.e. that proteasome inhibitors do not have any direct effect on virus particles or on the cell membrane (Example 1) and do not affect RNA replication, either (Example 3), led to the hypothesis that either the entry/internalization process or the uncoating, or else the assembly and secretion of progeny viruses, had to be affected by inhibition of the cellular proteasome. In order to test this hypothesis, MDBK cells were infected with BVDV and cultured for a further 30 hours in special capillaries having a diameter of 200 μm and in the presence or absence of 10 μm MG132. The cells were then fixed chemically and processed for TEM. An analogous procedure was adopted with virus supernatants from proteasome inhibitor-treated infected monolayer cultures and from mock-treated controls: the corresponding virus-containing cell culture supernatants were transferred into the capillaries, sealed, fixed chemically and prepared for TEM. In the electron microscopic analysis, it became clear that a significantly smaller number of virus particles, as compared with the mock control, can be detected in cellular vesicles in infected cells after the latter had been treated with PIs (FIG. 9). Furthermore, the ratio of completely assembled to incompletely assembled virions was shifted significantly in favor of incompletely assembled virus particles in PI-treated cells. When comparing virus-containing supernatants obtained from PI-treated and mock-treated cells, it became clear that the morphology of the viruses secreted during PI treatment had changed markedly when compared with wild-type viruses (FIG. 9). These findings provide support for the idea that the inhibitory effect of proteasome inhibitors on an infection with Flaviviridae is directed against the processes of assembling and/or secreting the progeny viruses.

### Example 5

### Treating Flaviviridae-Infected Cell Cultures with Different Classes of Proteasome Inhibitors Drastically Reduces the Production of Progeny Viruses.

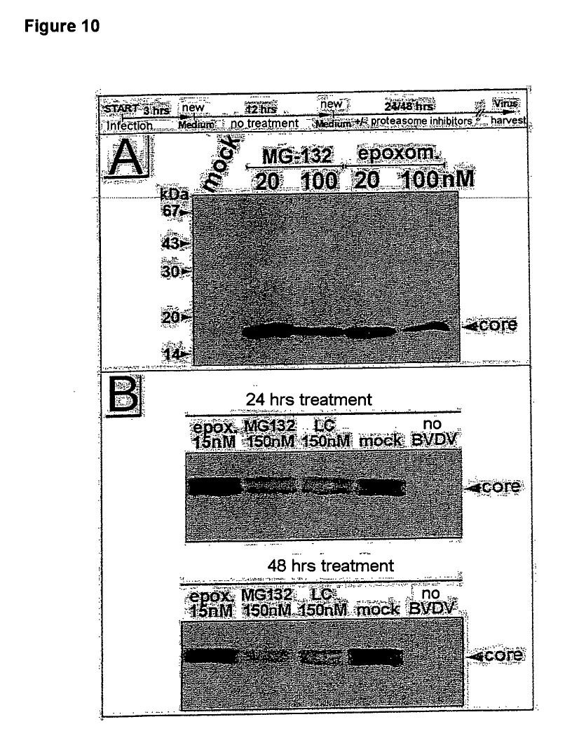

Parallel cultures of MDBK cells were infected, as described in the previous example, with equal MOIs of the pestivirus BVDV (CP7, NADL). 3 hours after infection, the cells were harvested, washed in PBS and cultured for a further 12 hours. This first washing step completely removed the input virus from the culture and, as a result, it is only possible to detect newly produced progeny viruses. After a further 12 hours of treatment, the cells were likewise harvested and washed; they were then aliquoted in equal cell quantities and treated with different proteasome inhibitors for 24 or 48 hours. After this period of treatment, the viruses were isolated by centrifuging them (99 min, 20,000×g, 4° C.) from identical volumes of cell culture supernatants. The period of time required in each case for infection, treatment and virus harvesting is shown in FIG. 10. The pelleted virions were denatured in SDS sample buffer and analyzed in a Western Blot using anti-BVDV core antibodies. In FIG. 10, part A, the Western blots were stained using antibodies which recognize the mature and processed BVDV core; in FIG. 10, part B, they were stained using antibodies which also recognize the immature, unprocessed core protein. In this connection, it can be clearly seen that an evident decrease occurs in the quantity of BVDV core protein, with this decrease depending on the dose of proteasome inhibitors. The effect of 100 nM epoxomicin was approximately comparable to the inhibitory effect of 200 nM of the peptide aldehyde MG132. Furthermore, the inhibition of the production of BVDV was augmented when the period of treatment was extended to 48 hours (FIG. 10, part B). For this, cells were treated with the proteasome inhibitors epoxomicin, MG132 and the beta-lactone lactacystin (FIG. 10, part B). In this connection, there was found to be a dose-dependent decrease in the core signal affecting both the mature form and the immature form of the BVDV core protein. As measured by the core signal, the quantity of BVDV virus decreased to 10% after 48 hours of treatment with 150 μM MG132 or lactacysteine (FIG. 10, part B). In agreement with the previous examples, this example shows, as a further finding, that chemically different classes of proteasome inhibitors block the production of BVDV virions. It was possible to demonstrate this directly by biochemically analyzing pelleted virions. Since this effect was demonstrated for proteasome inhibitors of the peptide aldehyde (MG132), epoxy ketone (epoxymicin), peptide vinyl sulfone (NLVS, data not shown) and lactone (LC) classes, it can consequently be assumed that the 26S proteasome is required for producing BVDV and thus other representatives of the Flaviviridae and that proteasome inhibitors are able to block the replication of Flaviviridae.

Legends to the Figures

FIG. 1: Effect of PIs on a Flaviviridae infection

Part 1: Effect of pretreating cells with PIs on a subsequent infection with Flaviviridae.

BHK-21 cells or MDBK cells were cultured, for 20 hours, at a concentration of 10 nM epoxomycin (E), lactacystin (L) or the proteasome inhibitor (Pi) MG132 in the culture medium. After the cells had been washed with PBS, they were infected with West Nile virus (BHK-21 cells) or BVDV CP7 (MDBK cells) at an MOI of 1-5. The titer, and the specific infectivity, of the progeny viruses present in the culture supernatant (see Application Example 2 for protocols) were determined at 12 and 30 hours after the infection.

The figure shows the titer of infectious West Nile virus and BVDV progeny viruses which were released after infecting PI pretreated, as compared with mock-pretreated, MDBK or BHK cells (values given as the PI-pretreated/mock-pretreated ratio in %; mock-pretreated=100%).

The figure furthermore shows the specific infectivity of the progeny viruses (values once again given as the PI-pretreated/mock-pretreated ratio in %; mock-pretreated=100%). The values shown correspond to the mean values from three independent experiments (standard deviation approx. 10%).

FIG. 2: Effect of PIs on a Flaviviridae infection

Part 2: Consequences of Pretreating West Nile Viruses and BVDV-CP7 Viruses with PIs on their Infectivity.

West Nile virus-containing or BVDV-containing culture supernatants were obtained from infected cells and the virus titers were determined (see Application Example 2 for protocols). 1 ml volumes of the supernatants were in each case adjusted to a concentration of 10 nM of a different PI (epoxomycin, lactacystin and MG132). The PI-treated supernatants, and a corresponding mock control, were incubated at 37° C. for 30 hours (cell culture conditions) and then used for infecting BHK cells (West Nile virus) and MDBK cells (BVDV). The titer and the specific infectivity of the resulting progeny viruses were determined using the protocols described under Application Example 2.

The figure shows the titers of infectious BVDV and West Nile virus progeny viruses released from MDBK and BHK cells, respectively, which had previously been infected either with PI-treated or mock-treated virus supernatants (values given as the PI-pretreated/mock-pretreated ratio in %; mock-pretreated=100%). The figure furthermore shows the specific infectivity of the progeny viruses (values once again given as the PI-pretreated/mock-pretreated ratio in %; mock-pretreated=100%). The values given correspond to mean values from three independent experiments (standard deviation approx. 10%).

FIGS. 3-5. Effect of PIs on a Flaviviridae infection

Part 3: Effect of a PI Treatment on Previously Infected Cells.

BHK-21 and MDBK cells were infected with BVDV or West Nile virus (see Application Example 2 for protocols) and, directly following the infection, the culture medium was adjusted to a concentration of 10 nM of different PIs (epoxomycin (E), lactacystin (L), MG132 (Pi)). After a CPE had been established, as a rule 12 hours (West Nile virus) or 30 hours (BVDV) after the infection, the titer and the specific infectivity of the progeny viruses which had been released were determined. The replication rate of the viral RNAs in the infected cells was also determined (protocols explained in Application Example 2).

Titers of infectious West Nile and BVDV progeny viruses which were released from BHK and, respectively, MDBK cells which has been treated, after the infection, with the given concentrations, and for the given times, with different proteasome inhibitors (see FIGS. 1 and 2). The figure shows the mean values from three independent virus titrations using in each case independent mock controls (standard deviation approx. 20%).

Specific infectivity of the progeny viruses as compared with the mock-treated controls, given as the PI-treated/mock-treated ratio in % (mock-treated=100%). The figure shows the mean values from three independent experiments (standard deviation approx. 20%).

Replication rate of the viruses as determined by RNase protection of the newly synthesized positive-strand RNA (Grassmann et al., 1999) given as the PI-treated/mock-treated ratio in % (mock-treated=100%). The figure shows the mean values from three independent experiments (standard deviation approx. 10%).

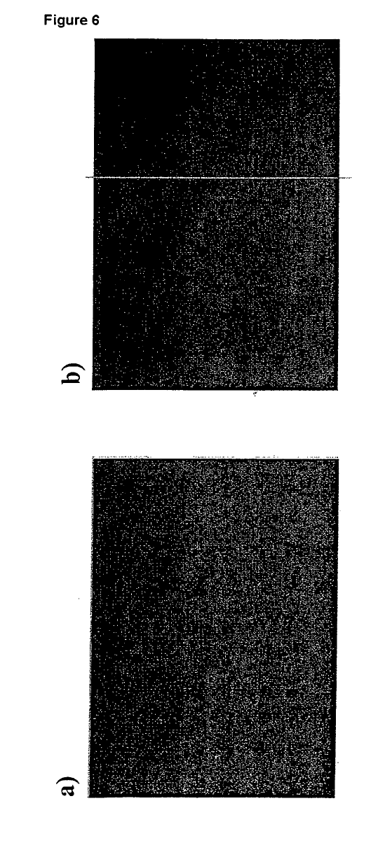

FIG. 6 Effect of treating Flaviviridae-infected cells with PIs on the spread of the infection in cell culture.

MBDK cells were infected with BVDV CP7, in conformity with the protocols explained in detail in Implementation Example 2, and treated with the proteasome inhibitor PI. 30 hours after the infection, the proteasome inhibitor-treated cells and, for comparison, cells which were untreated and otherwise infected under identical conditions, were subjected to an immunofluorescence test using anti-BVDV-NS3 antibody (Behrens et al., 1998). The figure shows the cells which were visualized by the immunofluorescence of the viral NS3 protein (microscopically enlarged 100-fold) in a) the absence of the proteasome inhibitor and b) following treatment with 10 nM of the proteasome inhibitor MG132 (Pi).

FIGS. 7 and 8. Effect of a PI treatment on the ability of BVDV and HCV replicon RNA to replicate

Organization of bicistronic BVDV and HCV RNA replicons as compared with the organization of the total BVDV and HCV genomes. Horizontal lines denote the untranslated regions (UTRs); boxes symbolize the translated regions of the viral RNAs. The nature of the proteolytic activity which leads to cleavage at the marked sites in the polyprotein is indicated by a corresponding symbol below the cleavage site. “Het. gene” symbolizes the additional gene (e.g. hygromycin B phosphotransferase) which is expressed in the bicistronic RNAs. For reasons to do with processing the N terminus of NS2-3, the ncp BVDV replicon also contains a part of the P7-encoding region. A part region of the N^{pro} gene (N-terminal BVDV autoprotease) and a part region of the capsid gene (in the case of HCV) are used for improving the efficiency of the IRES-mediated translation of the polyprotein. A ubiquitin gene (“Ubi”) was in some cases inserted in order to generate the authentic N terminus of the proteins: in this way, the proteolytic processing of the polyprotein at this site is effected using the cellular ubiquitin carboxyterminal hydrolase (Behrens et al., 1998; for a review, see Lindenbach and Rice, 2001; further explanations in the text).

Homogeneous MDBK and Huh-7 cell lines, which persistently contained the BVDV replicon and the HCV replicon, respectively, were treated for at least 30 hours with the different proteasome inhibitors epoxmycin (E) and lactacystin (L) and the proteasome inhibitor MG132 (Pi). Following this, the cells were detached from the culture plate and the cytoplasmic RNA was extracted using the methods described in Application Examples 2 and 3. Newly synthesized viral positive-strand RNA was detected and quantified by means of RNase protection. The figure compares the quantity of viral RNA which is extracted from PI-treated cells with that extracted from mock-treated cells: the figure shows the ratio of the quantity of viral RNA obtained from GI-treated cells to the quantity of viral RNA obtained from mock-treated cells in % (mock-treated RNA=100%). The values given are the mean values from three independent experiments (standard deviation approx. 10%). Immunofluorescence tests gave results which were in agreement with this (not shown).

FIG. 9 Electron microscopic studies of PI-treated and mock-treated MDBK cells following infection with BVDV

MDBK cells were infected with BVDV CP7 in accordance with the protocol explained under Application Example 4 and analyzed by electron microscopy.

FIG. 10:

Different Classes of PI Block the Production of BVDV CP7.

MDBK cells were infected with BVDV (CP7, NADL) in conformity with the previous application examples. In order to completely remove the input virus, the cells were washed and then further treated for 12 hours in order to enable the infection to become completely established. After they had been washed once again, the cells were treated for 24 or 48 hours with different concentrations of the proteasome inhibitors MG132, epoxomycin or lactacystin (LC). The viruses which were released were harvested and analyzed in a Western blot using anti-BVDV core antibodies which recognize either only mature core protein (A) or unprocessed core protein as well (B).

List of abbreviations

| | |
|---|---|
| ATP | adenosine 5′-triphosphate |
| BHK | baby hamster kidney |
| BVDV | bovine viral diarrhea virus |
| cDNA | copy DNA |
| CFTR | cystic fibrosis transmembrane regulator |
| CSFV | classical swine fever virus |
| Da | dalton (measure of molecular weight) |
| DHBV | duck hepatitis B virus |
| DNA | deoxyribonucleic acid |
| DTT | dithiothreitol |
| EDTA | ethylene diamine tetraacetic acid |
| EM | electron microscopy |
| EMCV | encephalomyocarditis virus |
| FDA | fluorescein diacetate |
| FSME | early summer meningoencephalitis |
| GB viruses | hepatitis GB viruses |
| HAART | highly active antiretroviral therapy |
| HAV | hepatitis A virus |
| HBV | hepatitis B virus |
| HCC | hepatocellular carcinoma |
| HCV | hepatitis C virus |
| HDV | hepatitis delta virus |
| HEPES | N-[2-hydroxyethyl]piperazine-N′-[2- |
| | ethanesulfonic acid] |
| HEV | hepatitis E virus |
| HFV | hepatitis F virus |
| HGV | hepatitis G virus |
| HIV | human immunodeficiency virus |
| HPV | human papillomavirus |
| Huh-7 | human transformed hepatoma cells |
| IFN | interferon |
| IL | interleukin |
| IRES | internal ribosomal entry site |
| kb | kilobases |
| IκB | inhibitory factor IκB |
| kDa | kilodalton (measure of molecular weight) |
| Ki | inhibitory constant |
| LC | lactacystin |
| LDL | low density lipoprotein |
| MDa | megadalton |
| MDBK | Marbin Darby bovine kidney |
| MHC | major histocompatibility complex |
| μM | micromolar |
| MG132 | protease inhibitor “MG132” |
| mM | millimolar |
| MOI | multiplicity of infection = number of |
| | infectious virus particles used per cell |
| NF-κB | transcription factor |
| NLVS | 4-hydroxy-5-iodo-3-nitrophenylacetyl-L- |
| | leucinyl-L-leucinyl-L-leucinevinylsulfone |
| nM | nanomolar |
| ncp | noncytopathic |
| ORF | open reading frame |
| PBS | phosphate buffer, phosphate-buffered |
| | saline |
| PCR | polymerase chain reaction |
| PGPH | postglutamyl peptide-hydrolyzing |
| PNGase | peptide N-glycosidase |
| PI | proteasome inhibitor(s) |
| PK | proteinase K |
| PS | ProScript |
| PS-341 | N-pyrazinecarbonyl-L-phenylalanine-L- |
| | leucine-boric acid |
| PS-519 | 1R-[1S,4R,5S]]-1-(1-hydroxy-2- |
| | methylpropyl)-4-propyl-6-oxa-2- |
| | azabicyclo[3.2.0]heptane-3,7-dione |
| PS-273 | morpholine-CONH—(CH—naphthyl)—CONH—(CH— |
| | isobutyl)-B)OH)₂ |
| PSI | N-carbobenzoxy-Ile-Glu(OBut)-Ala-Leu-H |
| RdRp | RNA-dependent RNA polymerase |
| RNA | ribonucleic acid |
| RNase | ribonuclease |
| RT | reverse transcriptase |
| SDS | sodium dodecyl sulfate |
| SDS-PAGE | SDS polyacrylamide gel electrophoresis |
| TEM | transmission electron microscopy |
| TNF | tumor necrosis factor |
| Tris | Tris buffer - |
| | tris(hydroxymethyl)aminomethane |
| Ub | ubiquitin |
| UCH | ubiquitin carboxyhydrolase |
| UPS | ubiquitin-proteasome system |
| UTR | untranslated region(s) (UTRs) |
| zLLL | N-carboxybenzoxyl-L-leucinyl-L-leucinyl- |
| | L-leucinal |
| cp | cytopathic |
| CPE | cytopathic effect of the virus infection |

### REFERENCES

Adams, J; Behnke, M; Chen, S; Cruickshank, A A; Dick, R L; Grenier, L; Klunder, J M; Ma, Y.-T; Plamondon, L; Stein, R L (1998). Potent and selective inhibition of the proteasome: dipeptidyl boronic acids. Bioorg. Med. Chem. Lett. 8:333-338.
Adams, J; Palombella, V J; Sausville, E A; Johnson, J; Destree, A; Lazarus, D D; Maas, J; Pien, C S; Prakash, S; Elliott, P J (1999). Proteasome inhibitors: a novel class of potent and effective antitumor agents. Cancer Res. 59:2615-2622.
Adams, J; Palombella, V J; Elliot, P J (2000). Proteasome inhibition: a new strategy in cancer treatment. Investigational New drugs 18:109-121.
Adams, J; Stein, R (1996). Novel inhibitors of the proteasome and their therapeutic use in inflammation. Annu. Rep. Med. Chem. 31:279-288.
Agnello, V, Abel, G, Elfahal, M, Knight, G B, Zhang, Q X; (1999). Hepatitis C virus and other flaviviridae viruses enter cells via low density lipoprotein receptor. Proc Natl Acad Sci USÃ. 96:12766-71.
Agnello, V; (2000). Mixed crypglobulinemia amd other extrahepatic manifestations of hepatitis C virus infections. In: Liang, T J, Hoonagle J H, eds. Hepatitis C. San Diego: Academic Press: 295-314.
Baldwin, A S (1996). The NF-kappa B and I kappa B proteins: new discoveries and insights. Annu. Rev. Immunol. 14:649-683.
Barton, D J, Flanegan J B; (1993). Coupled translation and replication of poliovirus RNA in vitro: synthesis of functional 3D polymerase and infectious virus. J. Virol. 67: 822-831.
Behrens, S E, Grassmann, C W, Thiel, H J, Meyers, G, Tautz, N; (1998). Characterization of an autonomous subgenomic RNA-replicon of a pestivirus. J. Virol. 72: 2364-2372.
Bukh, J, Miller R H, Purcell, R H; (1995). Genetic heterogeneity of hepatitis C virus: quasispecies and genotypes. Sem. Liver Dis. 15: 41-63.
Burke, D S, Monath, T P; (2001). Flaviviruses. In: Virology, fourth edition edited by B. N. Fields, Lippincott-Raven Philadelphia, New York: 1043-1125.
Ciechanover, A (1998). The ubiquitin-proteasome pathway: on protein death and cell life. EMBO J. 17:7151-7160.
Ciechanover, A, Orian, A, Schwartz, A L; (2000). The ubiquitin-mediated proteolytic pathway: mode of action and clinical implications. J. Cell Biochem. Suppl. 34: 40-51.
Coux, O; Tanaka, K; Goldberg, A L (1996). Structure and functions of the 20S and 26S proteasomes. Annu. Rev. Biochem. 65:801-847.
Dubuisson, J, Hsu, H H, Cheung, R C, Greenberg, H B, Russell, D G, Rice, C M; (1994). Formation and intracellular localization of hepatitis C virus envelope glycoprotein complexes expressed by recombinant vaccinina and Sindbis virus. J. Virol. 68: 6147-6160.
Elliott, P J, Ross J S; (2001). The proteasome: a new target for novel drug therapies. Am J Clin Pathol. 116:637-46.
Elliott, P J; Pien, C S; McCormack, T A; Capman, I D; Adams, J (1999). Proteasome inhibition: a novel mechanism to combat asthma. J. Allergy Clin. Immunol. 104:294-300.
Fenteany, G; Standaert, R F; Lane, W S; Choi, S; Corey, E J; Schreiber, S L (1995). Inhibition of proteasome activities and subunit-specific amino-terminal threonine modification by lactacystein. Science 268: 726-731.
Frankel, A; Man, S; Elliott, P; Adams, J; Kerbel, R S (2000). Lack of multicellular drug resistance observed in human ovarian and prostate carcinoma treated with the proteasome inhibitor PS-341. Clinical Cancer Res. 6:3719-3728.
Fuerst, T R, Niles, E G, Studier, F W, Moss B; (1986). Eukaryotic transient-expression system based on recombinant vaccinia virus that synthisizes bacteriophage T7 RNA polymerase. Proc. Natl. Acad. Sci. USA 83: 8122-8126.
Grassmann, C W, Isken, O, Behrens S E; (1999). Assignment of the multifunctional NS3 protein of bovine viral diarrhea virus during RNA replication: an in vivo and in vitro study. J. Virol. 73:9196-9205.
Grassmann, C W, Isken, O, Tautz, N, Behrens S E; (2001). Genetic analysis of the pestivirus nonstructural coding region: defects in the NS5A unit can be complemented in trans. J. Virol. 75: 7791-7802.
Hanada, M; Sugawara, K; Kaneta, K; Toda, S; Nishiyama, Y; Tomita, K; Yamamoto, H; Konishi, M; Oki, T (1992). Epoxomicin, a new antitumor agent of microbial origin. J. Anitbiot. (Tokyo) 45:1746-1752.
Harty, R N; Brown, M E; Wang, G; Huibregtse, J; Hayes, F P; (2000). A PPxY motif within the VP40 protein of Ebola virus interacts physically and functionally with a ubiquitin ligase: implications for filovirus budding. Proc. Natl. Acad. Sci. USA 97:13871-13876.
Hershko, A; Ciechanover, A; (1998). The ubiquitin system. Annu. Rev. Biochem. 67:425-479.
Higashitsuji, H; Itoh, K; Nagao, T; Dawson, S; Nonoguchi, K; Kido, T; Mayer, R J; Arii, S; Fujita, J; (2000). Reduced stability of retinoblastoma protein by gankyrin, an oncogenic ankyrin-repeat protein overexpressed in hepatomas. Nat Med. 6:96-99.
Köck J, Nassal M, MacNelly S, Baumert T F, Blum H E, von Weizsäcker F; (2001). Efficient infection of primary Tupaia hepatocytes with purified human and woolly monkey hepatitis B virus. J. Virol. 75:5084-5089.
Lohmann, V, Körner, F, Koch, J O, Herian, U, Theilmann, L., Bartenschlager R; (1999). Science 285:110-113.
Lightcap, E S; McCormack, T A; Pien, C S; Chau, V; Adams, J; Elliott, P J (2000). Proteasome inhibition measurements: clinical application. Clin. Chem. 46:673-683.
Lindenbach, B D, Rice, C M; (2001). Flaviviridae: the viruses and their replication. In: Virology, fourth edition edited by B. N. Fields, Lippincott-Raven Philadelphia, New York: 991-1042.
Major, M E, Rehermann, B, Feinstone, S M; (2001). Hepatitis C viruses. In: Virology, fourth edition edited by B. N. Fields, Lippincott-Raven Philadelphia, New York: 1127-1161.
Meng, L; Kwok, B H; Sin, N; Crews, C M (1999a). Eponemycin exerts its antitumor effect through the inhibition of proteasome function. Cancer Res. 59:2798-2801.
Meng, L; Mohan, R; Kwok, B H; Elofsson, M; Sin, N; Crews, C M (1999b). Epoxomicin, a potent and selective proteasome inhibitor, exhibits in vivo anti-inflammatory activity. Proc. Natl. Acad. Sci. USA. 96 (18): 10403-10408.
Merola, M, Brazzoli, M, Cochiarella, F, Heile, J M, Helenius, A, Weiner, A J, Houghton, M, Abri-gnani, S; (2001). Folding of Hepatitis C Virus E1 glycoprotein in a cell-free system. J. Virol. 75:11205-11217.
Meyers, G, Tautz, N, Becher, P, Thiel, H-J, Kümmerer B M; (1996). Recovery of cytopathogenic and noncytopathogenic bovine viral diarrhea viruses from cDNA contructs. J. Virol. 70:8606-8613.
Moormann, R J M, van Gennip, H G P, Miedema, G K L, Hulst, M M, van Rijn P A; (1996). Infectious RNA transcribed from an engineered full-length cDNA template of the genome of a pestivirus. J. Virol. 70:7-63-770.
Moradpour, D; Grabscheid, B; Kammer, A R; Schmidtke, G; Gröttrup, M; Blum, H E; Cerny, A (2001). Expression of hepatitis C virus proteins does not interfere with major histocompatibility complex class I processing and presentation in vitro. Hepatology 33:1282-1287.
Müller, K, Heimann, M, Rümenapf, T; (2000). Evidences that CD46 is involved in BVDV infection. Keystone symposium: Cell biology of virus entry, replication and pathogenesis. Taos, N M, Abstract supplement.
Palombella, V J; Conner, E M; Fuseler, J W; Destree, A; Davis, J M; Laroux, F S; Wolf, R E; Huang, J; Brand, S; Elliott, P J; Lazarus, D; McCormack, T; Parent, L; Stein, R; Adams, J; Grisham, M B; (1998). Role of the proteasome and NF-kappaB in streptococcal cell wall-induced polyarthritis. Proc. Natl. Acad. Sci. USA 95:15671-15676.
Palombella, V J; Rando, O J; Goldberg, A L; Maniatis, T; (1994). The ubiquitin-proteasome pathway is required for processing the NF-kappa B1 precursor protein and the activation of NF-kappa B. Cell 78:773-785.
Pamer, E; Cresswell, P; (1998). Mechanisms of MHC class I-restricted antigen processing. Annu. Rev. Immunol. 16:323-358.
Patnaik, A, Chau, V, Wills, J W; (2000). Ubiquitin is part of the retrovirus budding machinery. Proc. Natl. Acad. Sci. USA 97:13069-13074.
Pietschmann, T, Lohmann, V, Kaul, A, Krieger, N, Rinck, G, Rutter, G, Strand, D, Bartenschlager, R; (2002) Persistent and transient replication of full-length hepatitis C virus genomes in cell culture. J. Virol. 76:4008-21.
Phillips, J B; Williams, A J; Adams, J; Elliott, P J; Tortella, F C (2000). Proteasome inhibitor PS-519 reduces infarction and attenuates leukocyte infiltration in a rat model of focal cerebral ischemia. Stroke 31:1686-1693.
Rehermann, B; Ferrari, C; Pasquinelli, C; Chisari, F V (1996). The hepatitis B virus persists for decades after patients' recovery from acute viral hepatitis despite active maintenance of a cytotoxic T-lymphocyte response. Nat Med. 2:1104-1108.
Rock, K L; Gramm, C; Rothstein, L; Clark, K; Stein, R; Dick, L; Hwang, D; Goldberg, A L (1994). Inhibitors of the proteasome block the degradation of most cell proteins and the generation of peptides presented on MHC class I molecules. Cell 78:761-771.
Rock, K L; Goldberg, A L (1999). Degradation of cell proteins and the generation of MHC class I-presented peptides. Annu. Rev. Immunol. 17:739-779.
Rümenapf, T; Unger, G., Strauss, J H, and Theil, H J; (1993). Processing of the envelope glycoproteins of pestiviruses. J. Virol. 67: 3288-3294.
Schubert, U; Antón, L C; Gibbs, J; Norbury, C C; Yewdell, J W; Bennink, J R (2000b). Rapid degradation of a large fraction of newly sythesized proteins by proteasomes. Nature 404:770-774.
Schubert, U; Ott, D E; Chertova, E N; Welker, R; Tessmer, U; Princiotta, M F; Bennink, J R; Krasslich, H-G; Yewdell, J W (2000a). Proteasome inhibition interferes with gag polyprotein processing, release and maturation of HIV-1 and HIV-2. Proc Natl. Acad. Sci. USA 97:13057-13062.
Schwartz, A L; Ciechanover, A (1999). The ubiquitin-proteasome pathway and pathogenesis of human diseases. Annu. Rev. Med. 50:57-74.
Strack, B; Calistri, A; Accola, M A; Palu, G; Göttlinger, H G (2000). A role for ubiquitin ligase recruitment in retrovivus release. Proc. Natl. Acad. Sci. USA 97:13063-13068.
Suzuki, R; Tamura, K; Li, J; Ishii, K; Matsuura, Y; Miyamura, T; Suzuki, T (2001). Ubiquitin-mediated degradation of hepatitis C virus core protein is regulated by processing at its carboxyl terminus. Virology 280:301-309.
Teicher, B A; Ara, G; Herbst, R; Palombella, V J; Adams, J (1999). The proteasome inhibitor PS-341 in cancer therapy. Clin. Cancer Res. 5:2638-2645.
Thiel, H J, Plagemann, P G W, Moennig, V; (1996). Pestiviruses. In B. N. Fields, D. M. Knipe and P. M. Howley (ed.), Fields virology. Raven Press, Philadelphia. Pa.: 1059-1074.
Trautwein, C; Manns, M (2001). Antivirale Therapie der chronischen Virushepatitis [Antiviral therapy of chronic viral hepatitis]. Internist 42:913-923.
Yagi, T; Hardin, J A; Valenzuela, Y M; Miyoshi, H; Gores, G J; Nyberg, S L (2001). Caspase inhibition reduces apoptotic death of cryopreserved porcine hepatocytes. Hepatology 33:1432-40.
Yu, H, Grassmann, C W, Behrens S E; (1999). Sequence and structural elements at the 3′ terminus of bovine viral diarrhea virus genomic RNA: functional role during RNA replication. J. Virol. 73: 3638-3648.
Yu, H, Isken, O, Grassmann, C W, Behrens S E; (2000). A stem-loop motif formed by the immediate 5′-terminus of the bovine viral diarrhea virus genome modulates translation as well as replication of the viral RNA. J. Virol. 74:5825-5835.
Zhao, X, Tang Z, Klumpp B, Wolff-Vorbeck, G, Barth H, Levy S, von Weizsäcker, F, Blum H E, Baumert T F; (2002). Primary hepatocytes of Tupaia belangeri as a potential model for hepatitis C virus infection J. Clin. Invest. 109:221-232.

1-43. (canceled)

44. An agent for inhibiting at least one of release, maturation and replication of a member of the Flaviviridae family selected from Flavivirus, Pestivirus and Hepacivirus wherein the agent comprises, as an active component, at least one proteasome inhibitor in a pharmaceutical preparation.

45. An agent as claimed in claim 44, wherein the agent inhibits at least one of release, maturation and replication of hepatitis C virus (HCV) and is used for the treatment and prophylaxis of HCV-induced hepatitides, flavivirus-induced fever, hemorrhages, leukopenia, thrombocytopenia, diarrheal diseases and encephalitides and also pestivirus-induced diseases.

46. An agent as claimed in claim 45, wherein the proteasome inhibitor is a substance which inhibits, regulates or otherwise affects the activities of the ubiquitin/proteasome pathway; which specifically affects the enzymic activities of the complete 26S proteasome complex; and which specifically affects the enzymic activities of the free 20S, catalytically active, proteasome complex, which is not assembled with regulatory subunits.

47. An agent as claimed in claim 45, wherein the proteasome inhibitor is taken up by higher eukaryotic cells and, after having been taken up into a cell, interacts with the catalytic subunits of the proteasome, and, in connection with this, blocks at least some of the proteolytic activities of the proteasome within the 26S or the 20S proteasome complex.

48. An agent as claimed in claim 45, wherein in addition to proteasome inhibitors, the pharmaceutical preparation also comprises at least one further agent which affects, regulates or inhibits the cellular ubiquitin system, such as the activities of the ubiquitin-conjugating enzymes and/or of the ubiquitin-hydrolyzing enzymes.

49. An agent as claimed in claim 45, wherein the proteasome inhibitor is administered in various forms in vivo, i.e. orally, intravenously, intramuscularly, subcutaneously or in encapsulated form, with or without cell specificity-carrying changes, which, due to using a particular administration and/or dose regime, exhibit low cytotoxicity, which do not elicit any side effects, or only elicit insignificant side effects, and which exhibit a relatively high metabolic half life and a relatively low clearance rate in the body.

50. An agent as claimed in claim 45, wherein the proteasome inhibitor a) is isolated in natural form from microorganisms or other natural sources; or
b) is formed from natural substances as a result of chemical modifications; or
c) is prepared completely synthetically; or
d) is synthesized in vivo using gene therapy methods.

51. An agent as claimed in claim 50, wherein the proteasome inhibitor belongs to the following substance classes:
a) naturally occurring proteasome inhibitors:
peptide derivatives which contain epoxyketone structures C-terminally,
β-lactone derivatives,
aclacinomycin A (also termed aclarubicin),
lactacystin and its chemically modified variants, such as the cell membrane-penetrating variant “clastolactacystein β-lactone”
b) synthetically prepared proteasome inhibitors:
modified peptide aldehydes, such as N-carbobenzoxy-L-leucinyl-L-leucinyl-L-leucinal (also designated MG132 or zLLL), its boric acid derivative MG232; N-carbobenzoxy-Leu-Leu-Nva-H (designated MG115; N-acetyl-L-leucinyl-L-leucinyl-L-norleucinal (designated LLnL) and N-carbobenzoxy-Ile-Glu(OBut)-Ala-Leu-H (also designated PSI);
c) peptides which carry an α,β-epoxy ketone structure C-terminally, and also vinylsulfones, such as carbobenzoxy-L-leucinyl-L-leucinyl-L-leucine-vinylsulfone, or 4-hydroxy-5-iodo-3-nitrophenylacetyl-L-leucinyl-L-leucinyl-L-leucinevinylsulfone (NLVS)
d) glyoxylic acid or boric acid radicals, such as pyrazyl-CONH(CHPhe)CONH(CHisobutyl)B(OH)₂) and also dipeptidyl boric acid derivatives, or
e) pinacol esters, such as benzyloxycarbonyl(Cbz)-Leu-Leu-boroLeu pinacol ester.

52. An agent as claimed in claim 50 wherein the particularly suitable proteasome inhibitor is the epoxyketone epoxomicin (epoxomycin, molecular formula: C₂₈H₈₆N₄O₇) and/or eponemicin (eponemycin, molecular formula: C₂₀H₃₆N₂O₅).

53. An agent as claimed in claim 50 wherein the particularly suitable proteasome inhibitor is selected from the PS series
a) PS-519 as β-lactone, and also as lactacystin derivative the compound IR-[1S,4R,5S]]-1-(1-hydroxy-2-methylpropyl)-4-propyl-6-oxa-2-azabicyclo[3.2.0]heptane-3,7-dione-molecular formula C₁₂H₁₉NO₄— and/or
b) PS-341 as peptidyl-boric acid derivative the compound N-pyrazinecarbonyl-L-phenylalanine-L-leucine-boric acid—molecular formula C₁₉H₂₅BN₄O₄— and/or
c) PS-273 (morpholine-CONH—(CH-naphthyl)-CONH—(CH-isobutyl)-B(OH)₂) and its enantiomer PS-293 and/or
d) compound PS-296 (8-quinolylsulfonyl-CONH—(CH-naphthyl)-CONH(—CH-isobutyl)-B(OH)₂) and/or
e) PS-303 (NH₂(CH-naphthyl)-CONH—(CH-isobutyl)-B(OH)₂) and/or
f) PS-321 as (morpholine-CONH—(CH-naphthyl)-CONH—(CH-phenylalanine)-B(OH)₂); -and/or
g) PS-334 (CH₃—NH—(CH-naphthyl-CONH—(CH-isobutyl)-B(OH)₂) and/or
h) the compound PS-325 (2-quinol-CONH—(CH-homo-phenylalanine)-CONH—(CH-isobutyl)-B(OH)₂) and/or
i) PS-352 (phenylalanine-CH₂—CH₂-CONH—(CH-phenylalanine)-CONH—(CH-isobutyl)1-B(OH)₂) and/or
j) PS-383 (pyridyl-CONH—(CHpF-phenylalanine)-CONH—(CH-isobutyl)-B(OH)₂).

54. The use of proteasome inhibitors as claimed in claim 44 for inhibiting at least one of the entry/internalization process, the replication and the maturation and release of Flaviviridae.

55. The use of proteasome inhibitors as claimed in claim 54 for inhibiting late processes in the Flaviviridae life cycle.

56. The use as claimed in claim 54, wherein the proteasome inhibitor blocks to a large extent or completely prevent the production of infectious virions from Flaviviridae-infected cells.

57. The use as claimed in claim 54, wherein the proteasome inhibitor causes inhibition of the release of virions and also reduces the infectivity of the virions which are released.

58. The use as claimed in claim 54, wherein the proteasome inhibitor suppresses virus replication and thus the spread of an infection in vivo, i.e. in the liver tissue of an infected patient in the case of hepatitis C virus.

59. The use of proteasome inhibitors as claimed in claim 54 for inhibiting the replication of Flaviviridae in accordance with the following mechanisms
a) blocking/reducing the release of new virions;
b) blocking/reducing the infectivity of released virions;
c) blocking/reducing the spread of infection in cultures of host cells;
d) blocking/reducing the spread of infection in infected organs in vivo.

60. The use of proteasome inhibitors as claimed in claim 54 for suppressing flavivirus infections and pestivirus infections in humans and animals.

61. The use of proteasome inhibitors as claimed in claim 54 for inducing the death of hepatocarcinoma cells.

62. The use of proteasome inhibitors as claimed in claim 61 for suppressing and/or preventing the development of liver cell carcinomas.

63. The use of proteasome inhibitors as claimed in claim 62 for treating patients who have established liver cell carcinomas.

64. The use of proteasome inhibitors as claimed in claim 61 for treating/controlling/preventing HCV-induced liver cirrhosis and/or HCV-induced liver cell carcinomas, medicament-induced liver carcinomas, genetically determined liver carcinomas, environmentally determined liver carcinomas and/or liver carcinomas which are determined by a combination of viral and nonviral factors.

65. The use of proteasome inhibitors as claimed in claim 61 for selectively eliminating liver carcinoma cells which develop as the result of a HCV infection, or a corresponding coinfection with HCV and hepatitis B virus (HBV), or a hepatitis delta virus (HDV)/HBV/HCV coinfection, human immunodeficiency virus (HIV)/HCV coinfections, or HCV and coinfections with other viruses, bacteria or parasites.

66. The use of proteasome inhibitors as claimed in claim 61 for preventing the development, growth and metastasis of liver cell tumors and for preferentially destroying liver carcinoma cells in HCV-infected patients.

67. The use of proteasome inhibitors as claimed in claim 54 for modulating the expression, modification and activity of the tumor suppressor protein p53 and other tumor suppressor proteins which are of importance in connection with hepatocellular carcinomas (HCCs).

68. The use of proteasome inhibitors as claimed in claim 54 for liver cell regeneration in patients suffering from hepatitis.

69. The use of proteasome inhibitors as claimed in claim 54 for regenerating patients following flavivirus infections.

70. The use of proteasome inhibitors as claimed in claim 54 for regenerating stabled animals following flavivirus or pestivirus infections.

71. The use of proteasome inhibitors as claimed in claim 54 for reducing the number of infected virus-producing cells in liver cell tissue.

72. The use as claimed in claim 54 wherein the proteasome inhibitors alter the post-translational modification and proteolytic processing of Flaviviridae structural proteins and reduce the ability of the virus envelope proteins to dimerize and thereby reduce or block the release and infectivity of Flaviviridae.

73. The use of proteasome inhibitors as claimed in claim 54 for inhibiting both the maintenance and persistence of a previously established infection and of a secondary infection including blocking the spread of a Flaviviridae infection in vivo.

74. The use of more than one proteasome inhibitor as claimed in claim 50 in combination for the purpose of treating and controlling HCV-induced hepatitides, flavivirus-induced fever, hemorrhages and encephalitides and pestivirus-induced diseases.

75. The use as claimed in claim 74 in combination with therapeutic agents which are already used in the antiviral therapy of Flaviviridae infections.

76. The use as claimed in claim 74 for treating coinfections with different flaviviruses and pestiviruses.

77. The use as claimed in claim 74 for treating coinfections of HCV and immunodeficiency viruses HIV-1 and HIV-2.

78. The use as claimed in claim 77 for treating HCV/HIV coinfections in combination with HAART therapy.

79. The use of proteasome inhibitors as claimed in claim 54 for preventing a reinfection with HCV in connection with liver transplantations and other organ transplantations.

80. The use of proteasome inhibitors as claimed in claim 54 for preventing a reinfection with HCV in connection with cell therapies, by means of administering the agents before, during and after the transplantation.

81. The use of proteasome inhibitors as claimed in claim 54 for preventing a reinfection with HCV in connection with the transplantation of virus-free organs to chronic virus carriers who still possess residual virus and can infect new organs and also in connection with the transfer of virus-containing organs from donors to virus-free patients.

82. The use of proteasome inhibitors as claimed in claim 54 for preventing the establishment of a systemic Flaviviridae infection immediately following contact with infectious virus.

83. The use of proteasome inhibitors as claimed in claim 54 for preventing a Flaviviridae infection in individuals who are at a high risk of fresh infection.

84. The use of proteasome inhibitors as claimed in claim 54 for decreasing or eliminating a hepatitis by means of immune system-mediated mechanisms.

85. The use of proteasome inhibitors as claimed in claim 50 for producing agents and/or pharmaceutical preparations for inhibiting the release, maturation and replication of Flaviviridae.

86. The use of proteasome inhibitors as claimed in claim 85 for producing pharmaceuticals for the treatment and prophylaxis of HCV-induced hepatitides, flavivirus-induced fever, hemorrhages and encephalitides and pestivirus-induced diseases.
